(19) United States
(12) Patent Application Publication
(10) Pub. No.: US 2024/0093303 A1
(43) Pub. Date: Mar. 21, 2024

(54) METHODS AND BIOMARKERS IN CANCER

(71) Applicants: The Regents of the University of California, Oakland, CA (US); New York University, New York, NY (US)

(72) Inventors: Scott M. Lippman, La Jolla, CA (US); Webster K. Cavenee, La Jolla, CA (US); Teresa Davoli, New York, NY (US)

(21) Appl. No.: 18/264,314

(22) PCT Filed: Feb. 4, 2022
(86) PCT No.: PCT/US2022/015363

§ 371 Date: Aug. 4, 2023

(51) Int. Cl. C12Q 1/6886

(52) U.S. Cl. CPC C12Q 1/6886; C12Q 2600/106; C12Q 2600/156; C12Q 2600/158

(57) ABSTRACT

The disclosure further provides diagnostic, prognostic and therapeutic methods, which are based, at least in part, on determination of the identity of a genotype of interest identified herein.

### CROSS-REFERENCE TO RELATED APPLICATIONS

This application claims the benefit under 35 U.S.C. § 119(e) of U.S. Provisional Application Ser. No. 63/146,191, filed Feb. 5, 2021 and 63/179,215, filed Apr. 24, 2021, the contents of each of which are hereby incorporated by reference in its entirety.

### STATEMENT OF GOVERNMENT SUPPORT

This invention was made with government support under CA196408, TR001881, CA106451, CA097007, DE026644, CA023100, CA212621, and CA248631 awarded by the National Institutes of Health. The government has certain rights in the invention.

### BACKGROUND

The genetic bases for predisposition, and neoplastic transformation, to cancer have been increasingly well-described. However, it remains less clear how early, precancer and cancer cells employ these genetic alterations to acquire the characteristic features and properties (1) of malignant disease. For example, studies of the immune landscape led to breakthrough trials of programmed death-1 (PD-1) inhibitors for recurrent, metastatic head and neck squamous cell carcinoma (HNSC) therapy (2-4). This underscores the importance of immune modulation in these tumors, despite a still suboptimal overall response rate of less than 20% in advanced cancers. Immune response within tumors has been reported to be strongest at the earliest neoplastic stages, as recently shown in lung adenocarcinoma precursors (5). As such, new, immune-based strategies could be developed to reduce the high global burden of cancer such as for example HNSC (6-8). This disclosure satisfies this need and provides related advantages as well.

### SUMMARY OF THE DISCLOSURE

Applicant reports herein that somatic copy-number alterations (SCNAs) contribute to an immune hot-to-cold microenvironment switch in cancers, such as during HPV-negative head and neck cancer (HNSC) development. Specific and non-specific SCNA levels were examined in a large prospective oral precancer patient cohort, 3 HNSC patient cohorts and 32 cell lines. 9p21 loss in precursor lesions, the genomic driver of malignant transition, was enhanced by cumulative 9p-arm gene-dosage decreases, cell-intrinsic senescence suppression, decreases in three other pro-immunogenic pathways and immunoregulatory molecule imbalances. Furthermore, 9p-arm loss and JAK2-PD-L1 co-deletion (at 9p24) were associated with PD-1-inhibitor resistance. These data resolve the immune paradox, reveal an aneuploid checkpoint, and fuel distinct interception and therapeutic strategies for HNSC and possibly other tumors or aneuploid diseases.

Thus, in one aspect, this disclosure provides a method for treating cancer in a subject in need thereof, the method comprising, or consisting essentially of, or yet further consisting of, administering PD-1/PD-L1 centered immunotherapy to the subject if a sample isolated from the patient comprising the cancer cell does not show 9p-arm loss. Also provided is a method for treating cancer in a subject in need thereof, the method comprising, or alternatively consisting essentially of, or yet further consisting of, administering a therapy to the subject that does not comprise PD-1/PD-L1 centered immunotherapy to the subject if a sample isolated from the patient comprising the cancer cell shows 9p-arm loss.

Also provided are methods for treating cancer in a subject in need thereof, the method comprising, or alternatively consisting essentially of, or yet further consisting of, measuring gene deletion of JAK2 and PD-L1 in a cancer cell from the subject; wherein when genetic studies of JAK2 and PD-L1 is not indicative of co-deletion of JAK2 and PD-L1, then the subject is administered PD-1/PD-L1 centered immunotherapy, and wherein when genomic assessment of JAK2 and PD-L1 is indicative of co-deletion of JAK2 and PD-L1, then the subject is not administered PD-1/PD-L1 centered immunotherapy, and an alternative treatment is administered to the subject.

Further provided are methods of inhibiting the growth or progression of cancer in a subject comprising, or alternatively consisting essentially of, or yet further consisting of, i) measuring 9p-arm and 9p21 loss in a precancer cell from the subject; ii) determining a degree of 9p-arm loss; and iii) administering to the subject treatment that does not comprise PD-1/PD-L1 centered immunotherapy if the degree of 9p-arm loss shows significant 9p-arm loss.

Alternatively, an embodiment is provided for treating cancer in a subject in need thereof, the method comprising, or consisting essentially of, or yet further consisting of, i) measuring 9p-arm loss in a cancer cell from the subject; ii) determining a degree of 9p-arm loss; and iii) administering PD-1/PD-L1 centered immunotherapy if the degree of 9p-arm loss does not show significant 9p-arm loss.

In one aspect, methods for predicting or determining whether a subject with cancer will respond to treatment with immunotherapy is provided, the method comprising, or consisting essentially of, or yet further consisting of, i) measuring 9p-arm loss in a cancer cell from the subject; ii) determining a degree of 9p-arm loss; and iii) determining that the subject will respond to administering PD-1/PD-L1 centered immunotherapy if the degree of 9p-arm loss does not show significant 9p-arm loss.

Also provided are kits for carrying out the disclosed methods, the kits comprising instructions to carry out the methods, and optionally, instructions to carry out the methods.

### BRIEF DESCRIPTION OF THE DRAWINGS

FIGS. 1A-1E: Precancer SCNA and immune-cell infiltrate associations: (FIG. 1A) Distributions of CD3+ and CD8+ cells by 3p14, 9p21.3, and 17p13.1 loss and p-values for comparing the two groups for each covariate using the linear mixed-effect models on log-transformed data. (FIG. 1B) Distributions of CD3+, CD8+ and CD68+ cells by any loss of 3p14, 9p21, 17p13.1 and p-values for comparing the two groups for each covariate using the linear mixed-effect models on log-transformed data. (FIGS. 1C-1E) Coefficients of estimates (standard error) and p-values from multivariable logistic regression models of CD3+, CD8+, and CD68+ cells dichotomized based on top and bottom 35% of the distribution. Each model includes 3p14, 9p21.3 or 17p13.1 loss and SCNA level as independent variables. A positive coefficient indicates that patients with the SCNA event were more likely to have higher CD3+, CD8+ and CD68+ cell levels than those without the event. Findings were confirmed by dichotomizing the parameters based on median, in addition to top and bottom 35%, of the distribution.

FIGS. 2A-2D: HPV-negative HNSC SCNA and immune-cell infiltrate associations: (FIG. 2A) Relationship between expression level of CD3 (CD3D gene) and CD8 (CD8A gene) with CN loss (arm or focal) at 9p21.3, 3p14 or 17p13.1. p-value is from Wilcoxon test. (FIG. 2B) Multivariable logistic regression model for the prediction of CD3+ and CD8+ cell levels. Variable importance (size effect) is also shown. (FIG. 2C) Multivariable logistic regression model for the prediction of CD68+ cells. (FIG. 2D) Multivariable logistic regression similar to (FIG. 2B) but considering 9p (arm) loss, 9p21.3 (focal) loss, and 9p21.3 (arm & focal) loss as separate variables.

FIGS. 3A-3E: SASP, JAK/STAT, Cytokine receptor, TNFA signaling via NFKB Pathways in relationship with 9p or 9p21.3 loss in HPV-negative HNSC cell lines: (FIG. 3A) Pathway depletion from GSEA analysis of HPV-negative HNSC cell lines comparing lines with and without 9p loss. GSEA plots, NES (normalized enrichment score), p-value, and FDR are shown for and SASP, JAK/STAT, Cytokine-cytokine receptor, and TNFA signaling via NFKB pathways. (FIGS. 3B-3E) Relationship between gene expression of the SASP (FIG. 3B), JAK/STAT (FIG. 3C), Cytokine-cytokine receptor pathway (FIG. 3D), and TNFA Signaling via NFkB (FIG. 3E) (ssGSEA analysis) and 9p arm or 9p21.3 loss. p-value is from Wilcoxon test.

FIGS. 4A-4C: Chromosome 9p loss in ICB (FIG. 4A) and (FIG. 4B) non-ICB treated HPV-negative HNSC; focal 9p21 loss in ICB treated patients (FIG. 4C).

FIGS. 5A-5B: Immune hot-to-cold aneuploid switch model of oral precancer-to-cancer transition: (FIG. 5A) In precancer, the dominant CN driver of immune-hot lesions was chromosome gain (trisomy, tetrasomy) and 9p21 loss (right). Immunogenic precancers with SCNAs must acquire other genomic alterations (see FIG. 5B) in order to overcome an aneuploid checkpoint, or barrier (center), that then leads to immune evasion (CD3/CD8+ T-cell depletion) (left) during the precancer-to-cancer transition. Tumors with complex karyotypes and other key, associated genomic events, notably TP53 mutation or loss (associated with aneuploidy in human tumors) (21) or downstream pathway inactivation (69) could mask SASP, cGAS-STING, or other cell-intrinsic responses triggered by aneuploid cells (center, right of aneuploid checkpoint), and shed light on the apparent opposing roles of these immune regulatory pathways during tumorigenesis (see below. (FIG. 5B) Possible genetic mechanisms for the aneuploid switch involving early loss of chromosome 9p21 and augmentation or expansion of deletion size in later stages of HPV-negative head and neck tumorigenesis. Right top, increased deletion size in cancer; right center, expanded 9p-arm loss to encompass neighboring regulatory or cooperating gene deletion. The genes involved depend on whether the deletion expansion is restricted to 9p21 (and include CDKNT2A), is telomeric (in this illustration, could include JAK2 on 9p24), centromeric (and so could include SASP-related cytokine-cytokine pathway genes in 9p13 (data not shown), and/or involves epistatic interaction between 9p21 loss and events elsewhere in the cancer genome (in this example, inactivating mutation of the TP53 gene on 17p13) thereby creating synthetic physiological effects, that exacerbate phenotype severity (55, 56). Without being bound by theory, aneuploid diseases may reflect complex interactions of key driver genes with combined sum effects of a large number of interacting, CN altered genes (47). In the latter context, the number of genes encoded on an altered chromosome is directly proportional to the severity of the SCNA phenotype. Chromosome loss regions are indicated in dark blue and delineated by adjacent solid black lines. Mutation is indicated with a red “X”.

FIGS. 6A-6C: Predictors of cancer risk in prospective oral precancer cohort: (FIG. 6A) Kaplan-Meier curves of oral cancer-free survival (OCFS) by SCNAs (9p21.3, 3p14 or 17p13.1 loss, no chromosomal loss). (FIG. 6B) Kaplan-Meier curves of OCFS by combination of SCNA with histology status (dysplasia and 9p21.3 loss versus others). (FIG. 6C) Multivariable Cox-proportional hazard model for prediction of survival (OCFS).

FIG. 7: CD3+(CD3D), CD8+(CD8A) or CD68 expression and IS in HNSC.

FIGS. 8A-8C: SCNA associations with CD3+, CD8+, GZMB/IFNg or IS in HNSC: CD3+, CD8+ and GZMB/IFNg (average expression of the two genes) or IS associations with the presence of any loss (arm or focal) at 9p21.3, 3p14, or 17p13 (FIG. 8A), SCNA level (FIG. 8B), or chr7 gain (FIG. 8C).

FIGS. 9A-9D: 3p14, 9p21.3 or 17p.13.1 loss (arm or focal) associated with SCNA level in HNSC: (FIG. 9A) Relationship between the number of arm loss (loss_arm), gain (gain_arm) and SCNA level (loss_arm+gain_arm, cnv_Arm) between samples with and without 3p14 loss (arm or focal). (FIG. 9B) Arm loss, gain and SCNA level associations with and without 9p21.3 loss. (FIG. 9C) Arm loss, gain and SCNA level associations with or without 17p13.1 loss. (FIG. 9D)) Arm loss, gain and SCNA level associations with or without loss (arm or focal) at any of the three sites (3p14, 9p21.3 or 17p13.1) (p-value is from Wilcoxon test).

FIG. 10: Percentage of loss on arm 9p in HNSC (based on cytoband regions): Representative genes indicated.

FIGS. 11A-11D: Chromosome-9p loss and CD8+ T-cell associations with HNSC stage and TP53 status: Association between CD8+ T-cell levels and 9p loss (FIG. 11A) and 9p21.3 loss (FIG. 11B), in relationship with tumor stage and TP53 mutation status (FIG. 11C and FIG. 11D) in HPV-negative stage I or II HNSC tissue samples.

FIGS. 12A-12D: SCNA level to different HNSC tumor stage: (FIG. 12A) SCNA level and CD8+ T cells in relation to tumor stage. (FIG. 12B) SCNA level and CD3+ T cells in relation to tumor stage. (FIG. 12C) SCNA level and CD8 T cells (via CIBERSORT) in relation to tumor stage. (FIG. 12D) SCNA level and NK cells (via CIBERSORT) in relation to tumor stage.

FIGS. 13A-13D: Chromosome 9p loss and CD3+ T-cell associations with HNSC stage and TP53: Association between CD3+ T-cell levels and 9p loss (FIG. 13A) and 9p21.3 loss (FIG. 13B), in relationship with tumor stage and TP53 mutation status (FIG. 13C and FIG. 13D) in HPV-negative HNSC tissue samples, the latter focused on stage-I and -II samples.

FIGS. 14A-14E: SASP, JAK/STAT and Cytokine receptor and TNFA Signaling via NFkB Pathways in relationship with 9p or 9p21.3 loss in HPV-negative HNSC tumors: (FIG. 14A) Pathway depletion from GSEA analysis of HPV-negative HNSC tumors comparing samples with and without 9p loss. GSEA plots, NES (normalized enrichment score), p-value and FDR. (FIGS. 14B-14E) Relationship between the gene expression of the SASP (FIG. 14B), JAK/STAT (FIG. 14C), Cytokine-cytokine receptor pathway (FIG. 14D) and TNFA Signaling via NFkB (FIG. 14E), and 9p arm loss, 9p21.3 loss, or 9p21.3 “focal” loss in HNSC tumors. p-value is from Wilcoxon test.

FIGS. 15A-15D: IFNA gene set enrichment in HPV-negative cell lines or tumors with or without 9p or 9p21.3 loss: (FIGS. 15A-15D) Relationship between IFNα gene set enrichment (through ssGSEA) and 9p arm or 9p21.3 loss in HPV-negative cell lines (FIG. 15A, FIG. 15B) or tumors (FIG. 15C, FIG. 15D). p-value is from Wilcoxon test.

FIG. 16: Representative genes differentially expressed in tumors or cell lines with or without 9p loss: Relationship between the expression level of the indicated genes comparing HPV-negative cell lines or tumors with or without 9p arm-level. p-value is from Wilcoxon test.

FIG. 17: Consort diagram of Real-World Evidence (RWE) cohort: Cohort of HPV-negative HNSC patients treated with nivolumab or pembrolizumab (or chemotherapy) whose tumors had been profiled using a next generation sequencing platform.

### DETAILED DESCRIPTION

### Definitions

Throughout this disclosure, various publications, patents and published patent specifications are referenced by an identifying citation or by an Arabic numeral, the full citations for which are found immediately preceding the claims. The disclosures of these publications, patents and published patent specifications are hereby incorporated by reference into the present disclosure to more fully describe the state of the art to which this disclosure pertains.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

As used herein, the term “comprising” is intended to mean that the methods include the recited elements, but not excluding others. “Consisting essentially of” when used to define methods, shall mean excluding other elements of any essential significance to the method. “Consisting of” shall mean excluding more than trace elements of other ingredients for claimed compositions and substantial method steps. Embodiments defined by each of these transition terms are within the scope of this disclosure. Accordingly, it is intended that the methods can include additional steps and components (comprising) or alternatively including steps of no significance (consisting essentially of) or alternatively, intending only the stated method steps (consisting of).

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a cancer.

As used herein, the term “sample isolated from a subject” or “test sample” refers to any liquid or solid material containing nucleic acids. In suitable embodiments, a test sample is obtained from a biological source (i.e., a “biological sample”), such as cells in culture or a tissue sample from an animal, preferably, a human. In some embodiments, a biological sample comprises a sample selected from blood, serum, plasma, a throat swab, a nasal swab, a nasopharyngeal wash, saliva, urine, gastric fluid, cerebrospinal fluid, tears, stool, mucus, sweat, earwax, oil, a glandular secretion, semen, vaginal fluid, interstitial fluids derived from tumorous tissue, ocular fluids, breath, hair, finger nails, skin, biopsy tissue, placental fluid, amniotic fluid, cord blood, lymphatic fluids, cavity fluids, sputum, pus, microbiota, meconium, breast milk, and other secretions or excretions. In a specific embodiment, the sample is a biopsy sample.

The term “determining” or “identifying” is to associate or affiliate a patient closely to a group or population of patients who likely experience the same or a similar clinical response to a therapy.

As used herein, the term “deletion or loss of a genomic region” where X refers to 9p, any 9p cytoband (e.g. 9p21, 9p22, 9p13) or gene (e.g. MLLT3, ELAVL2, KLH9) as the presence of a genomic (DNA) copy number loss of the genomic region, using a cutoff between −0.15 and −0.3 for the log 2FC (log 2 fold change, where the fold change is the ratio between the copy number of the genomic region and the copy number of the rest of the genome). For example, if the copy number of 9p is 1 and the copy number of the rest of the genome is 2, the log 2FC is −1 and 9p is considered lost (see also Methods: PMID:33952700).

As used herein, the term “decrease or suppression in a gene level” intends the presence of a significant decrease in the RNA level of the gene (measured by RNA sequencing) assessed using the following method as compared to a normal or healthy level. In one aspect, this can be determined by splitting the samples into 2 groups, for example 9p loss and no 9p loss. DESeq2 (https://bioconductor.org/packages/release/bioc/vignettes/DESeq2/inst/doc/DESeq2.html) or compatible program is used to detect the gene-level differential expression comparing the 2 groups of samples. The deriving p-values are adjusted by Benjamin and Hochberg or equivalent method for FDR. In one aspect, a cutoff of 0.05 p-value or 0.1 FDR is used to consider a gene significantly differentially expressed. In the case of PD-L1 protein level, it is measured by immune histochemistry with antibodies 22C3 or 28-8 (see also Methods: PMID:33952700).

As used herein, the term “decrease or suppression in a pathway level” intends the presence of a significant decrease in the average RNA levels of the genes involved in the pathway (measured by RNA sequencing) based on a method called Gene Set Enrichment Analysis (https://www.gsea-msigdb.org/gsea/index.jsp) or equivalent method. Using the method disclosed above for “decrease in gene level” is performed and then run GSEA using the following score as the input: sign (log 2FoldChange)*−log 10 (adjusted p-value) where the log 2FC and the p-value for each gene are derived from the DESeq2 output. In one aspect, a cutoff of 0.05 p-value or 0.1 FDR is used to consider a pathway significantly differentially expressed (see also Methods: PMID:33952700).

The term “selecting” a patient for a therapy refers to making an indication that the selected patient is suitable for the therapy. Such an indication can be made in writing by, for instance, a handwritten prescription or a computerized report making the corresponding prescription or recommendation.

“Having the same cancer” is used when comparing one patient to another or alternatively, one patient population to another patient population. For example, the two patients or patient population will each have or be suffering from colon cancer.

A “normal cell corresponding to the tumor tissue type” refers to a normal cell from a same tissue type as the tumor tissue. A non-limiting example is a normal lung cell from a patient having lung tumor, or a normal colon cell from a patient having colon tumor.

“Detecting, measuring or assessing” as used herein refers to determining the presence of a nucleic acid or gene of interest in a sample or the presence of a protein of interest in a sample. Detection does not require the method to provide 100% sensitivity and/or 100% specificity.

“Detectable label” as used herein refers to a molecule or a compound or a group of molecules or a group of compounds used to identify a nucleic acid or protein of interest. In some cases, the detectable label can be detected directly. In other cases, the detectable label can be a part of a binding pair, which can then be subsequently detected. Signals from the detectable label can be detected by various means and will depend on the nature of the detectable label. Detectable labels can be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluorescence, or chemiluminescence, or any other appropriate means.

The terms “oligonucleotide” or “polynucleotide” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

When a genetic marker, e.g., SCNA, is used as a basis for selecting a patient for a treatment described herein, the genetic marker is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker in a clinical setting is a clear indication that this parameter was used as a basis for initiating, continuing, adjusting and/or ceasing administration of the treatments described herein.

Cancer, Staging and Types

In certain embodiments, the terms “disease” “disorder” and “condition” are used interchangeably herein, referring to a precancer or alternatively cancer, a status of being diagnosed with a cancer, or a status of being suspect of having a cancer. “Cancer”, which is also referred to herein as “tumor”, is a known medically as an uncontrolled division of abnormal cells in a part of the body, benign or malignant. Non-limiting examples of malignant neoplasms include a broad group of diseases involving unregulated cell division and growth, and invasion to nearby parts of the body. Non-limiting examples of cancers include carcinomas, sarcomas, leukemia and lymphoma, e.g., colon cancer, colorectal cancer, rectal cancer, gastric cancer, melanoma, non-small cell lung cancer, small cell lung cancer, esophageal cancer, head and neck cancer, HPV negative head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas, carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas. In aspect, the cancer or precancer is not renal cancer.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. In aspect, the cancer or precancer is not renal cancer.

Precancer cells or tumors are cells or tissue that contain abnormal cells that have an increased risk of turning cancerous.

Staging is the process of determining details about your cancer, such as tumor size and if it has spread. Typically, the stage guides treatment decisions. Stage I means the cancer is localized to the tissue where it originated. Stage II and III mean the cancer is larger and has grown into nearby tissues or lymph nodes. Stage IV indicates that the cancer cells are found in other organs from where the cancer originated.

In certain embodiments, the terms “disease” “disorder” and “condition” are used interchangeably herein, referring to a cancer, a status of being diagnosed with a cancer, or a status of being suspect of having a cancer. “Cancer”, which is also referred to herein as “tumor”, is a known medically as an uncontrolled division of abnormal cells in a part of the body, benign or malignant. In one embodiment, cancer refers to a malignant neoplasm, a broad group of diseases involving unregulated cell division and growth, and invasion to nearby parts of the body. Non-limiting examples of cancers include carcinomas, sarcomas, leukemia and lymphoma, e.g., head and neck cancer, melanoma, colon cancer, colorectal cancer, rectal cancer, gastric cancer, esophageal cancer, head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas, carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas. In aspect, the cancer or precancer is not renal cancer.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. As used herein, an advanced cancer refers to a cancer that had progressed after receiving one or more of: the first line therapy, the second line therapy, or the third line therapy.

Head and neck cancer (HNC) develops from tissues in the lip and oral cavity (mouth), the larynyx (throat), salivary glands, nose, sinuses or the skin of the face. The most common types of head and neck cancers occur in the lip, mouth, and larynx. HNC may be associated with prior infection with high-risk types of HPV (e.g., HPV-16 and -18) and is responsible for HPV-positive HNC. HPV positive and negative tumors represent a different clinicopathological and molecular entities. Many HPV-negative HNC are tobacco and alcohol inducted and are characterized by TP53 mutation. See http://atlasgeneticsoncology.org/Tumors/HeadNeckSCCID5078.html, last accessed on Jan. 29, 2022.

Cancer Therapies and Methods of Administration

The term “suitable for a therapy” or “suitably treated with a therapy” shall mean that the patient is likely to exhibit one or more desirable clinical outcomes as compared to patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide or alternatively, SCNA. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In further another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, can exhibit more than one more desirable clinical outcomes as compared to patients having the same disease and receiving the same therapy but not possessing the characteristic. As defined herein, the patient is considered suitable for the therapy. In another such aspect, a patient possessing a characteristic can exhibit one or more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

As used herein, the term “administration” and “administering” are used to mean introducing an agent into a subject. Routes of administration include, but are not limited to, oral (such as a tablet, capsule or suspension), topical, transdermal, intranasal, vaginal, rectal, subcutaneous intravenous, intravenous, intraarterial, intramuscular, intraosseous, intraperitoneal, intraocular, subconjunctival, sub-Tenon's, intravitreal, retrobulbar, intracameral, intratumoral, epidural and intrathecal.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

“Therapeutically effective amount” of a drug or an agent refers to an amount of the drug or the agent that is an amount sufficient to obtain a pharmacological response or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

When the disease is cancer, the following clinical endpoints are non-limiting examples of treatment: (1) elimination of a cancer in a subject or in a tissue/organ of the subject or in a cancer loci; (2) reduction in tumor burden (such as number of cancer cells, number of cancer foci, number of cancer cells in a foci, size of a solid cancer, concentrate of a liquid cancer in the body fluid, and/or amount of cancer in the body); (3) stabilizing or delay or slowing or inhibition of cancer growth and/or development, including but not limited to, cancer cell growth and/or division, size growth of a solid tumor or a cancer loci, cancer progression, and/or metastasis (such as time to form a new metastasis, number of total metastases, size of a metastasis, as well as variety of the tissues/organs to house metastatic cells); (4) less risk of having a cancer growth and/or development; (5) inducing an immune response of the patient to the cancer, such as higher number of tumor-infiltrating immune cell, higher number of activated immune cells, or higher number cancer cell expressing an immunotherapy target, or higher level of expression of an immunotherapy target in a cancer cell; (6) higher probability of survival and/or increased duration of survival, such as increased overall survival (OS, which may be shown as 1-year, 2-year, 5-year, 10-year, or 20-year survival rate), increased progression free survival (PFS), increased disease free survival (DFS), increased time to tumor recurrence (TTR) and increased time to tumor progression (TTP). In some embodiments, the subject after treatment experiences one or more endpoints selected from tumor response, reduction in tumor size, reduction in tumor burden, increase in overall survival, increase in progression free survival, inhibiting metastasis, improvement of quality of life, minimization of drug-related toxicity, and avoidance of side-effects (e.g., decreased treatment emergent adverse events). In some embodiments, improvement of quality of life includes resolution or improvement of cancer-specific symptoms, such as but not limited to fatigue, pain, nausea/vomiting, lack of appetite, and constipation; improvement or maintenance of psychological well-being (e.g., degree of irritability, depression, memory loss, tension, and anxiety); improvement or maintenance of social well-being (e.g., decreased requirement for assistance with eating, dressing, or using the restroom; improvement or maintenance of ability to perform normal leisure activities, hobbies, or social activities; improvement or maintenance of relationships with family). In some embodiments, improved patient quality of life that is measured qualitatively through patient narratives or quantitatively using validated quality of life tools known to those skilled in the art, or a combination thereof. Additional non-limiting examples of endpoints include reduced hospital admissions, reduced drug use to treat side effects, longer periods off-treatment, and earlier return to work or caring responsibilities. In one aspect, prevention or prophylaxis is excluded from treatment.

Administration or treatment in “combination” refers to administering two agents such that their pharmacological effects are manifest at the same time. Combination does not require administration at the same time or substantially the same time, although combination can include such administrations.

The phrase “first line” or “second line” or “third line” etc., refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as primary therapy and primary treatment.” See National Cancer Institute website as www.cancer.gov, last visited on May 1, 2008. Typically, a patient is given a subsequent chemotherapy regimen because the patient did not shown a positive clinical or sub-clinical response to the first line therapy or the first line therapy has stopped.

The term “chemotherapy” or encompasses cancer therapies that employ chemical or biological agents or other therapies, such as radiation therapies, e.g., a small molecule drug or a large molecule, such as antibodies, Chimeric antigen receptor (CAR) therapies, RNAi and gene therapies. Non-limiting examples of chemotherapeutic agents are provided below. Unless specifically excluded, when a specific therapy is recited, equivalents of the therapy are within the scope of this disclosure.

An “immunotherapy agent” means a type of cancer treatment which uses a patient's own immune system to fight cancer, including but not limited to a physical intervene, a chemical substance, a biological molecule or particle, a cell, a tissue or organ, or any combinations thereof, enhancing or activating or initiating a patient's immune response against cancer. Non-limiting examples of immunotherapy agents include antibodies, immune regulators, checkpoint inhibitors, an antisense oligonucleotide (ASO), a RNA interference (RNAi), a Clustered Regularly Interspaced Short Palindromic Repeat (CRISPR) system, a viral vector, an anti-cancer cell therapy (e.g., transplanting an anti-cancer immune cell optionally amplified and/or activated in vivo, or administering an immune cell expressing a chimeric antigen receptor (CAR)), a CAR therapy, and cancer vaccines. As used herein, unless otherwise specified, an immunotherapy agent is not an inhibitor of thymidylate biosynthesis, or an anthracycline or other topoisomerase II inhibitor. As used herein, immune checkpoint refers to a regulator and/or modulator of the immune system (such as an immune response, an anti-tumor immune response, a nascent anti-tumor immune response, an anti-tumor immune cell response, an anti-tumor T cell response, and/or an antigen recognition of T cell receptor in the process of immune response). Their interaction activates either inhibitory or activating immune signaling pathways. Thus a checkpoint may contain one of the two signals: a stimulatory immune checkpoint that stimulates an immune response, and an inhibitory immune checkpoint inhibiting an immune response. In some embodiments, the immune checkpoint is crucial for self-tolerance, which prevents the immune system from attacking cells indiscriminately. However, some cancers can protect themselves from attack by stimulating immune checkpoint targets. In some embodiments, the immune checkpoints are present on T cells, antigen-presenting cells (APCs) and/or tumor cells.

One target of an immunotherapy agent is a tumor-specific antigen while the immunotherapy directs or enhances the immune system to recognize and attack tumor cells. Non-limiting examples of such agent includes a cancer vaccine presenting a tumor-specific antigen to the patient's immune system, a monoclonal antibody or an antibody-drug conjugate specifically binding to a tumor-specific antigen, a bispecific antibody specifically binding to a tumor-specific antigen and an immune cell (such as a T-cell engager or a NK-cell engager), an immune cell (such as a killer cell) specifically binding to a tumor-specific antigen (such as a CAR-T cell, a CAR-NK cell, and a CAR-NKT cell), a polynucleotide (or a vector comprising the same) transfecting/transducing an immune cell to express an tumor-specific antibody of an antigen binding fragment thereof (such as a CAR), or a polynucleotide (or a vector comprising the same) transfecting/transducing a cancer cell to express an antigen or a marker which can be recognized by an immune cell.

Another exemplified target is an inhibitory immune checkpoint which suppresses the nascent anti-tumor immune response, such as A2AR, B7-H3, B7-H4, BTLA, CTLA-4, CTLA-4/B7-1/B7-2, IDO, KIR, LAG3, NOX2, PD-1, PD-L1 and TIM-3, VISTA, SIGLEC7 (Sialic acid-binding immunoglobulin-type lectin 7, also designated as CD328) and SIGLEC9 (Sialic acid-binding immunoglobulin-type lectin 9, also designated as CD329). Non-limiting examples of such agent includes an antagonist or inhibitor of an inhibitory immune checkpoint, an agent reducing the expression and/or activity of an inhibitory immune checkpoint (such as via an antisense oligonucleotide (ASO), a RNA interference (RNAi), or a Clustered Regularly Interspaced Short Palindromic Repeat (CRISPR) system), an antibody or an antibody-drug conjugate or a ligand specifically binding to and reducing (or inhibiting) the activity of an inhibitory immune checkpoint, an immune cell with reduced (or inhibited) an inhibitory immune checkpoint (and optionally specifically binding to a tumor-specific antigen, such as a CAR-T cell, a CAR-NK cell, and a CAR-NKT cell), and a polynucleotide (or a vector comprising the same) transfecting/transducing an immune cell or a cancer cell to reduce or inhibit an inhibitory immune checkpoint thereof. Reducing expression or activity of such inhibitory immune checkpoint enhances immune response of a patient to a cancer.

A further possible immunotherapy target is a stimulatory checkpoint molecule (including but not limited to 4-1BB, CD27, CD28, CD40, CD122, CD137, OX40, GITR and ICOS), wherein the immunotherapy agent actives or enhances the anti-tumor immune response. Non-limiting examples of such agent includes an agonist of a stimulatory checkpoint, an agent increasing the expression and/or activity of a stimulating immune checkpoint, an antibody or an antibody-drug conjugate or a ligand specifically binding to and activating or enhancing the activity of a stimulating immune checkpoint, an immune cell with increased expression and/or activity of a stimulating immune checkpoint (and optionally specifically binding to a tumor-specific antigen, such as a CAR-T cell, a CAR-NK cell, and a CAR-NKT cell), and a polynucleotide (or a vector comprising the same) transfecting/transducing an immune cell or a cancer cell to express a stimulating immune checkpoint thereof.

Additional or alternative targets may be utilized by an immunotherapy agent, such as an immune regulating agent, including but not limited to, an agent activating an immune cell, an agent recruiting an immune cell to a cancer or a cancer cell, or an agent increasing immune cell infiltrated into a solid tumor and/or a cancer loci. Non-limiting examples of such agent is an immune regulator or a variant, a mutant, a fragment, an equivalent thereof.

In some embodiments, an immunotherapy agent utilizes one or more targets, such as a bispecific T cell engager, a bispecific NK cell engager, or a CAR cell therapy. In some embodiments, the immunotherapy agent targets one or more immune regulatory or effector cells.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits, rat, canine, donkey, mice, camelids (such as dromedaries, llamas, and alpacas), as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴M⁻¹ greater or at least 10⁵ M⁻¹ greater than a binding constant for other molecules in a biological sample). The term “antibody” also includes genetically engineered forms such as chimeric antibodies (for example, murine or humanized non-primate antibodies), heteroconjugate antibodies (such as, bispecific antibodies). See also, Pierce Catalog and Handbook, 1994-1995 (Pierce Chemical Co., Rockford, Ill.); Owen et al., Kuby Immunology, 7^{th} Ed., W.H. Freeman & Co., 2013; Murphy, Janeway's Immunobiology, 8^{th} Ed., Garland Science, 2014; Male et al., Immunology (Roitt), 8^{th} Ed., Saunders, 2012; Parham, The Immune System, 4^{th} Ed., Garland Science, 2014. The term “antibody” includes any protein or peptide containing molecule that comprises at least a portion of an immunoglobulin molecule, such as the whole antibody and any antigen binding fragment or a single chain thereof. The terms “antibody,” “antibodies” and “immunoglobulin” also include immunoglobulins of any isotype, fragments of antibodies which retain specific binding to antigen, including, but not limited to, Fab, Fab′, F(ab)₂, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues. The antibodies can be polyclonal, monoclonal, multispecific (e.g., bispecific antibodies), and antibody fragments, so long as they exhibit the desired biological activity.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

In some embodiments, the antibody is a bispecific immune cell engager, referring to a bispecific monoclonal antibody that is capable of recognizing and specifically binding to a tumor antigen (such as CD19, EpCAM, MCSP, HER2, EGFR or CS-1) and an immune cell, and directing an immune cell to cancer cells, thereby treating a cancer. Non-limiting examples of such antibody include bispecific T cell engager, bispecific cytotoxic T lymphocytes (CTL) engager, and bispecific NK cell engager. In one embodiment, the engager is a fusion protein consisting of two single-chain variable fragments (scFvs) of different antibodies. Additionally or alternatively, the immune cell is a killer cell, including but not limited to: a cytotoxic T cell, a gamma delta T cell, a NK cell and a NK-T cell.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains.

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells for using in a cell therapy and/or a CAR therapy may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses.

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells for using in a cell therapy and/or a CAR therapy may either be isolated or obtained from a commercially available source.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity or side effect.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at cancer.gov. Typically, a patient is given a subsequent therapy because the patient did not shown a positive clinical or sub-clinical response to the first line therapy or the first line therapy has stopped.

The term “adjuvant” therapy refers to administration of a therapy or chemotherapeutic regimen to a patient in addition to the primary or initial treatment, such as after removal of a tumor by surgery. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure. Additionally or alternatively, such adjuvant therapy potentials (i.e., sensitizes the subject to the original therapy) the subject may help reach one or more of clinical endpoints of the cancer treatment.

An “immunotherapy agent” means a type of cancer treatment which uses a patient's own immune system to fight cancer, including but not limited to a physical intervene, a chemical substance, a biological molecule or particle, a cell, a tissue or organ, or any combinations thereof, enhancing or activating or initiating a patient's immune response against cancer. Non-limiting examples of immunotherapy agents include antibodies, immune regulators, checkpoint inhibitors, an antisense oligonucleotide (ASO), a RNA interference (RNAi), a Clustered Regularly Interspaced Short Palindromic Repeat (CRISPR) system, a viral vector, an anti-cancer cell therapy (e.g., transplanting an anti-cancer immune cell optionally amplified and/or activated in vivo, or administering an immune cell expressing a chimeric antigen receptor (CAR)), a CAR therapy, and cancer vaccines. As used herein, unless otherwise specified, an immunotherapy agent is not an inhibitor of thymidylate biosynthesis, or an anthracycline or other topoisomerase II inhibitor. As used herein, immune checkpoint refers to a regulator and/or modulator of the immune system (such as an immune response, an anti-tumor immune response, a nascent anti-tumor immune response, an anti-tumor immune cell response, an anti-tumor T cell response, and/or an antigen recognition of T cell receptor in the process of immune response). Their interaction activates either inhibitory or activating immune signaling pathways. Thus a checkpoint may contain one of the two signals: a stimulatory immune checkpoint that stimulates an immune response, and an inhibitory immune checkpoint inhibiting an immune response. In some embodiments, the immune checkpoint is crucial for self-tolerance, which prevents the immune system from attacking cells indiscriminately. However, some cancers can protect themselves from attack by stimulating immune checkpoint targets. In some embodiments, the immune checkpoints are present on T cells, antigen-presenting cells (APCs) and/or tumor cells.

One target of an immunotherapy agent is a tumor-specific antigen while the immunotherapy directs or enhances the immune system to recognize and attack tumor cells. Non-limiting examples of such agent includes a cancer vaccine presenting a tumor-specific antigen to the patient's immune system, a monoclonal antibody or an antibody-drug conjugate specifically binding to a tumor-specific antigen, a bispecific antibody specifically binding to a tumor-specific antigen and an immune cell (such as a T-cell engager or a NK-cell engager), an immune cell (such as a killer cell) specifically binding to a tumor-specific antigen (such as a CAR-T cell, a CAR-NK cell, and a CAR-NKT cell), a polynucleotide (or a vector comprising the same) transfecting/transducing an immune cell to express an tumor-specific antibody of an antigen binding fragment thereof (such as a CAR), or a polynucleotide (or a vector comprising the same) transfecting/transducing a cancer cell to express an antigen or a marker which can be recognized by an immune cell.

Another exemplified target is an inhibitory immune checkpoint which suppresses the nascent anti-tumor immune response, such as A2AR, B7-H3, B7-H4, BTLA, CTLA-4, CTLA-4/B7-1/B7-2, IDO, KIR, LAG3, NOX2, PD-1, PD-L1 and TIM-3, VISTA, SIGLEC7 (Sialic acid-binding immunoglobulin-type lectin 7, also designated as CD328) and SIGLEC9 (Sialic acid-binding immunoglobulin-type lectin 9, also designated as CD329). Non-limiting examples of such agent includes an antagonist or inhibitor of an inhibitory immune checkpoint, an agent reducing the expression and/or activity of an inhibitory immune checkpoint (such as via an antisense oligonucleotide (ASO), a RNA interference (RNAi), or a Clustered Regularly Interspaced Short Palindromic Repeat (CRISPR) system), an antibody or an antibody-drug conjugate or a ligand specifically binding to and reducing (or inhibiting) the activity of an inhibitory immune checkpoint, an immune cell with reduced (or inhibited) an inhibitory immune checkpoint (and optionally specifically binding to a tumor-specific antigen, such as a CAR-T cell, a CAR-NK cell, and a CAR-NKT cell), and a polynucleotide (or a vector comprising the same) transfecting/transducing an immune cell or a cancer cell to reduce or inhibit an inhibitory immune checkpoint thereof. Reducing expression or activity of such inhibitory immune checkpoint enhances immune response of a patient to a cancer.

A further possible immunotherapy target is a stimulatory checkpoint molecule (including but not limited to 4-1BB, CD27, CD28, CD40, CD122, CD137, OX40, GITR and ICOS), wherein the immunotherapy agent actives or enhances the anti-tumor immune response. Non-limiting examples of such agent includes an agonist of a stimulatory checkpoint, an agent increasing the expression and/or activity of a stimulating immune checkpoint, an antibody or an antibody-drug conjugate or a ligand specifically binding to and activating or enhancing the activity of a stimulating immune checkpoint, an immune cell with increased expression and/or activity of a stimulating immune checkpoint (and optionally specifically binding to a tumor-specific antigen, such as a CAR-T cell, a CAR-NK cell, and a CAR-NKT cell), and a polynucleotide (or a vector comprising the same) transfecting/transducing an immune cell or a cancer cell to express a stimulating immune checkpoint thereof.

As used herein the term “PD-1” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the PD-1 sequence as shown herein and/or a suitable binding partner of PD-L1. Non-limiting example sequences of PD-1 are provided herein, such as but not limited to those under the following reference numbers—GCID:GC02M241849; HGNC: 8760; Entrez Gene: 5133; Ensembl: ENSG00000188389; OMIM: 600244; and UniProtKB: Q15116—and the sequence: MQIPQAPWPVVWAVLQLGWRPGWFLDSPDRPWNPPTFSPALLVVTEGDNATFTCSF SNTSESFVLNWYRMSPSNQTDKLAAFPEDRSQPGQDCRFRVTQLPNGRDFHMSVVR ARRNDSGTYLCGAISLAPKAQIKESLRAELRVTERRAEVPTAHPSPSPRPAGQFQTLV VGVVGGLLGSLVLLVWVLAVICSRAARGTIGARRTGQPLKEDPSAVPVF SVDYGEL DFQWREKTPEPPVPCVPEQTEYATIVFPSGMGT SSPARRGSADGPRSAQPLRPEDGHC SWPL, and equivalents thereof. Non-limiting examples of commercially available antibodies thereto include pembrolizumab (Merck), nivolumab (Bristol-Myers Squibb), pidilizumab (Cure Tech), AMP-224 (GSK), AMP-514 (GSK), PDR001 (Novartis), and cemiplimab (Regeneron and Sanofi).

As used herein the term “PD-L1” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the PD-L1 sequence as shown herein and/or an suitable binding partner of PD-1. Non-limiting example sequences of PD-L1 are provided herein, such as but not limited to those under the following reference numbers—GCID: GC09P005450; HGNC: 17635; Entrez Gene: 29126; Ensembl: ENSG00000120217; OMIM: 605402; and UniProtKB: Q9NZQ7—and the sequence: MRIFAVFIFMTYWHLLNAFTVTVPKDLYVVEYGSNMTIECKFPVEKQLDLAALIVY WEMEDKNIIQFVHGEEDLKVQHSSYRQRARLLKDQLSLGNAALQITDVKLQDAGVY RCMISYGGADYKRITVKVNAPYNKINQRILVVDPVTSEHELTCQAEGYPKAEVIWTS SDHQVLSGKTTTTNSKREEKLFNVTSTLRINTTTNEIFYCTFRRLDPEENHTAELVIPE LPLAHPPNERTHLVILGAILLCLGVALTFIFRLRKGRMMDVKKCGIQDTNSKKQSDT HLEET, and equivalents thereof. Non-limiting examples of commercially available antibodies thereto include atezolizumab (Roche Genentech), avelumab (Merck Soreno and Pfizer), durvalumab (AstraZeneca), BMS-936559 (Bristol-Myers Suibb), and CK-301 (Chekpoint Therapeutics).

Additional or alternative targets may be utilized by an immunotherapy agent, such as an immune regulating agent, including but not limited to, an agent activating an immune cell, an agent recruiting an immune cell to a cancer or a cancer cell, or an agent increasing immune cell infiltrated into a solid tumor and/or a cancer loci. Non-limiting examples of such agent is an immune regulator or a variant, a mutant, a fragment, an equivalent thereof.

In some embodiments, an immunotherapy agent utilizes one or more targets, such as a bispecific T cell engager, a bispecific NK cell engager, or a CAR cell therapy. In some embodiments, the immunotherapy agent targets one or more immune regulatory or effector cells.

A “tumor response” (TR) refers to a tumor's response to therapy. A “complete response” (CR) to a therapy refers to the clinical status of a patient with evaluable but non-measurable disease, whose tumor and all evidence of disease have disappeared following administration of the therapy. In this context, a “partial response” (PR) refers to a response that is anything less than a complete response. “Stable disease” (SD) indicates that the patient is stable following the therapy. “Progressive disease” (PD) indicates that the tumor has grown (i.e. become larger) or spread (i.e. metastasized to another tissue or organ) or the overall cancer has gotten worse following the therapy. For example, tumor growth of more than 20 percent since the start of therapy typically indicates progressive disease. “Non-response” (NR) to a therapy refers to status of a patient whose tumor or evidence of disease has remained constant or has progressed.

“Overall Survival” (OS) refers to the length of time of a cancer patient remaining alive following a cancer therapy.

“Progression free survival” (PFS) or “Time to Tumor Progression” (TTP) refers to the length of time following a therapy, during which the tumor in a cancer patient does not grow. Progression-free survival includes the amount of time a patient has experienced a complete response, partial response or stable disease.

“Disease free survival” refers to the length of time following a therapy, during which a cancer patient survives with no signs of the cancer or tumor.

“Time to Tumor Recurrence (TTR)” refers to the length of time, following a cancer therapy such as surgical resection or chemotherapy, until the tumor has reappeared (come back). The tumor may come back to the same place as the original (primary) tumor or to another place in the body.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

One chemotherapy is 5-Fluorouracil (5-FU) which belongs to the family of therapy drugs called pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluoroidine), 1-tetrahydrofuranyl-5-fluorouracil (ftorafur), capecitabine (Xeloda®), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamichael (1999) The Oncologist 4:478-487.

Cetuximab or Erbitux (commercially available from Lily) is an FDA-approved antibody to the epidermal growth factor receptor (EGFR) that is used alone or in combination with irinotecan (also known as CPT-11 or Camptosar) to treat various cancers. See https://chemocare.com/chemotherapy/drug-info/cetuximab.aspx.

Another chemotherapy is 5-FU based adjuvant therapy which refers to 5-FU alone or alternatively the combination of 5-FU with one or more other treatments, that include, but are not limited to radiation, methyl-CCNU, leucovorin, oxaliplatin (such as cisplatin), irinotecan, mitomycin, cytarabine, doxorubicin, cyclophosphamide, and levamisole, as well as an immunotherapy. Specific treatment adjuvant regimens are known in the art such as weekly Fluorouracil/Leucovorin, weekly Fluorouracil/Leucovorin+Bevacizumab, FOLFOX, FOLFOX-4, FOLFOX6, modified FOLFOX6 (mFOLFOX6), FOLFOX6 with bevacizumab, mFOLFOX6+Cetuximab, mFOLFOX6+Panitumumab, modified FOLFOX7 (mFOLFOX7), FOLFIRI, FOLFIRI with Bevacizumab, FOLFIRI+Ziv-aflibercept, FOLFIRI with Cetuximab, FOLFIRI+Panitumumab, FOLFIRI+Ramucirumab, FOLFOXIRI, FOLFIRI with FOLFOX6, FOLFOXIRI+Bevacizumab, FOLFOXIRI+Cetuximab, FOLFOXIRI+Panitumumab, Roswell Park Fluorouracil/Leucovorin, Roswell Park Fluorouracil/Leucovorin+Bevacizumab, Simplified Biweekly Infusional Fluorouracil/Leucovorin, Simplified Biweekly Infusional Fluorouracil/Leucovorin+Bevacizumab, and MOF (semustine (methyl-CCNU), vincrisine (Oncovin®) and 5-FU). For a review of these therapies see Beaven and Goldberg (2006) Oncology 20(5):461-470 as well as www.cancertherapyadvisor.com/home/cancer-topics/gastrointestinal-cancers/gastrointestinal-cancers-treatment-regimens/colon-cancer-treatment-regimens/. Other chemotherapeutics can be added, e.g., oxaliplatin or irinotecan.

Capecitabine is chemotherapy that is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

Leucovorin (Folinic acid) is a chemotherapy which is an adjuvant used in cancer therapy. It is used in synergistic combination with 5-FU to improve efficacy of the chemotherapeutic agent. Without being bound by theory, addition of Leucovorin is believed to enhance efficacy of 5-FU by inhibiting thymidylate synthase. It has been used as an antidote to protect normal cells from high doses of the anticancer drug methotrexate and to increase the antitumor effects of fluorouracil (5-FU) and tegafur-uracil. It is also known as citrovorum factor and Wellcovorin. This compound has the chemical designation of L-Glutamic acid N-[4-[[(2-amino-5-formyl-1,4,5,6,7,8-hexahydro-4-oxo-6-pteridinyl)methyl]amino]benzoyl], calcium salt (1:1).

“Oxaliplatin” (Eloxatin) is a chemotherapy that is a platinum-based chemotherapy drug in the same family as cisplatin and carboplatin. It is typically administered in combination with fluorouracil and leucovorin in a combination known as FOLFOX for the treatment of colorectal cancer. Compared to cisplatin, the two amine groups are replaced by cyclohexyldiamine for improved antitumor activity. The chlorine ligands are replaced by the oxalato bidentate derived from oxalic acid in order to improve water solubility. Equivalents to Oxaliplatin are known in the art and include, but are not limited to cisplatin, carboplatin, aroplatin, lobaplatin, nedaplatin, and JM-216 (see McKeage et al. (1997) J. Clin. Oncol. 201:1232-1237 and in general, Chemotherapy for Gynecological Neoplasm, Curr. Therapy and Novel Approaches, in the Series Basic and Clinical Oncology, Angioli et al. Eds., 2004).

“FOLFOX” is chemotherapy that is an abbreviation for a type of combination therapy that is used to treat cancer. This therapy includes leucovorin (“FOL”), 5-FU (“F”), and oxaliplatin (“OX”) and encompasses various regimens, such as FOLFOX-4, FOLFOX-6, modified FOLOX-6, and FOLFOX-7, which vary in doses and ways in which each of the three drugs are administered. “FOLFIRI” is an abbreviation for a type of combination therapy that is used treat cancer and comprises, or alternatively consists essentially of, or yet further consists of 5-FU, leucovorin, and irinotecan. Information regarding these treatments are available on the National Cancer Institute's web site, cancer.gov, last accessed on May 30, 2020 as well as www.cancertherapyadvisor.com/home/cancer-topics/gastrointestinal-cancers/gastrointestinal-cancers-treatment-regimens/colon-cancer-treatment-regimens/, last accessed on May 30, 2020.

Irinotecan (CPT-11) is a chemotherapy sold under the trade name of Camptosar. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation.

S-1 is a chemotherapy that consists of three agents (at a molar ratio of 1:0.4:1): tegafur, 5-chloro-2-4-dihydroxypyridine, and potassium oxonate.

An “antifolate” is a drug or biologic chemotherapy that impairs the function of folic acids, e.g., an antimetabolite agent that inhibits the use of a metabolite, i.e. another chemical that is part of normal metabolism. In cancer treatment, antimetabolites interfere with DNA production, thus cell division and growth of the tumor. Non-limiting examples of these agents are dihydrofolate reductase inhibitors, such as methotrexate, Aminopterin, and Pemetrexed; thymidylate synthase inhibitors, such as Raltitrexed or Pemetrexed; purine based, i.e. an adenosine deaminase inhibitor, such as Pentostatin, a thiopurine, such as Thioguanine and Mercaptopurine, a halogenated/ribonucleotide reductase inhibitor, such as Cladribine, Clofarabine, Fludarabine, or a guanine/guanosine: thiopurine, such as Thioguanine; or Pyrimidine based, i.e. cytosine/cytidine: hypomethylating agent, such as Azacitidine and Decitabine, a DNA polymerase inhibitor, such as Cytarabine, a ribonucleotide reductase inhibitor, such as Gemcitabine, or a thymine/thymidine: thymidylate synthase inhibitor, such as a Fluorouracil (5-FU).

### Descriptive Embodiments

The disclosure further provides diagnostic, prognostic and therapeutic methods, which are based, at least in part, on determination of the identity of a genotype of interest identified herein.

For example, information obtained using the diagnostic assays described herein is useful for determining if a subject is suitable for cancer treatment of a given type. Based on the prognostic information, a doctor can recommend a therapeutic protocol, useful for reducing the malignant mass or tumor in the patient or treat cancer in the individual.

A patient's likely clinical outcome following a clinical procedure such as a therapy or surgery can be expressed in relative terms. For example, a patient having a particular genotype or expression level can experience relatively longer overall survival than a patient or patients not having the genotype or expression level. The patient having the particular genotype or expression level, alternatively, can be considered as likely to survive. Similarly, a patient having a particular genotype or expression level can experience relatively longer progression free survival, or time to tumor progression, than a patient or patients not having the genotype or expression level. The patient having the particular genotype or expression level, alternatively, can be considered as not likely to suffer tumor progression. Further, a patient having a particular genotype or expression level can experience relatively shorter time to tumor recurrence than a patient or patients not having the genotype or expression level. The patient having the particular genotype or expression level, alternatively, can be considered as not likely to suffer tumor recurrence. Yet in another example, a patient having a particular genotype or expression level can experience relatively more complete response or partial response than a patient or patients not having the genotype or expression level. The patient having the particular genotype or expression level, alternatively, can be considered as likely to respond. Accordingly, a patient that is likely to survive, or not likely to suffer tumor progression, or not likely to suffer tumor recurrence, or likely to respond following a clinical procedure is considered suitable for the clinical procedure.

It is to be understood that information obtained using the diagnostic assays described herein can be used alone or in combination with other information, such as, but not limited to, genotypes or expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis or treatment.

The methods are useful in the assistance of an animal, a mammal or yet further a human patient. For the purpose of illustration only, a mammal includes but is not limited to a human, a simian, a murine, a bovine, an equine, a porcine or an ovine subject.

Genetic Mutations and Detection

As used herein, “9p21.3” (3p14 or 17p13.1) loss means either focal or arm loss (i.e., deletion at 9p21.3 region could derive from arm or focal events), unless specifically qualified as “focal only.”

The term “somatic copy-number alteration” (SCNA) intends an alteration in a gene copy number acquired by a cell that can be passed to the progeny of the mutated cell in the course of division. SCNA can be determined by methods known in the art. Non-limiting examples of such include fluorescent in situ hybridization, comparative genomic hybridization, array comparative genomic hybridization, single nucleotide polymorphism (SNP) array, genomic sequencing, high resolution microarray, and karyotype analysis. In one aspect, the SCNA is determined using a method comprising, consisting essentially of, or yet further consisting of SNP array.

### Modes for Carrying Out the Disclosure

Therapeutic and Prognostic Methods

This disclosure provides methods for several methods for treating cancer. In one embodiment, provided herein is a method for treating cancer in a subject in need thereof, the method comprising, or consisting essentially thereof, or consisting of administering PD-1/PD-L1 centered immunotherapy to the subject if a sample isolated from the patient comprising the cancer cell does not show 9p-arm loss. In another aspect, a method is provided for treating cancer in a subject in need thereof, the method comprising, or consisting essentially thereof, or yet further consisting of administering a therapy to the subject that does not comprise PD-1/PD-L1 centered immunotherapy to the subject if a sample isolated from the patient comprising the cancer cell shows 9p-arm loss. In a further aspect, the method comprises, or consists essentially of, or yet further consists of: i) measuring 9p-arm loss in a cancer cell from the subject; ii) determining a degree of 9p-arm loss; and iii) administering PD-1/PD-L1 centered immunotherapy if the degree of 9p-arm loss does not show significant 9p-arm loss.

The subject can be a mammal, e.g., a mammal such as a rat, mouse, canine, feline or human. When practiced in an animal, it can be used therapeutically or as an animal model to test new therapies and combination therapies. The sample can be as described herein, and will vary with the cancer being treated, e.g., a tumor biopsy or sample containing the cancer cell.

Methods and tools for measuring such therapeutic efficacy is known to one of skill in the art, including measuring a clinical endpoint in a human patient and/or in an animal/tissue/cell model mimicking a patient having a cancer. For example, therapeutic efficacy may be monitored by CT scan or blood work analysis. In addition, tumor markers may be assessed. In one aspect, treatment is determined by one or more longer overall survival, reduced tumor burden, longer time to tumor recurrence and inhibition of cancer progression.

In some embodiments, the cancer cell is a cell of a cancer selected from cancers of the: circulatory system, for example, heart (sarcoma [angiosarcoma, fibrosarcoma, rhabdomyosarcoma, liposarcoma], myxoma, rhabdomyoma, fibroma, lipoma and teratoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); gastrointestinal stromal tumors and neuroendocrine tumors arising at any site; genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Karposi's sarcoma, moles dysplastic nevi, lipoma, angioma, dermatofibroma, and keloids; and other tissues comprising connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites. Additionally or alternatively, the cancer is a solid tumor or a liquid cancer. In some embodiments, the cancer is a primary cancer. In another embodiment, the cancer is a metastasis. In aspect, the cancer or precancer is not renal cancer.

In some embodiments, the cancer cell is from a carcinoma, a sarcoma, a myeloma, a leukemia, or a lymphoma. In some embodiments, the cancer cell is from a carcinoma. In some embodiments, the cancer cell is from a sarcoma. In some embodiments, the cancer cell is from a myeloma.

Various methods can be used to assess the level of or significance of 9p-arm loss in the patient sample. In one aspect, the significance of 9-arm loss comprises assessing 9p21.3 loss. In one aspect wherein the cancer is HPV-HNSC and the significant 9p-arm loss comprises 9p21.3 loss, the assessment is associated with immune-cold tumor microenvironment (TME) signal.

In one aspect, a significant loss of 9p21.3 of the entire 9p arm measured as loss of ≥70% of arm length. In a further aspect, the loss comprises suppression of IFN-γ-inducible CXCL9/10 expression.

In a further aspect, the methods further comprise measuring CD8+ levels in a sample isolated from the patient and wherein immune cold TME signal is indicated by a low CD8+ level. In this aspect, the sample comprises a sample comprising CD8+ T cells and the levels are determined by the amount of transcripts of the genes CD8A and CD8B (average) measured by RNA sequencing.

In a further aspect, the 9p21.3 loss is associated with the presence of mutated TP53. TP53 mutational status is determined by the presence in the cancer cells of at least one mutation among the following types of mutation: nonsense, splice site, frame shift or missense with Polyphen score>0.2 (Polyphen score based on: PMID 20354512).

In a yet further aspect, 9p21.3 loss is associated with cell-intrinsic senescence-associated secretory pathway (SASP) suppression.

In one embodiment, 9p21.3 loss comprises deletion of one or more of the following genes: MLLT3; ELAVL2 or KLHL9.

9p-arm loss can also be measured by assessing 9p22.1 loss. In one aspect, 9p22.1 loss comprises deletion of one or more of the following genes: RPS6; DENND4C; RRAGA; or HAUS6.

9p-arm loss can also determined by 9p22.2 loss. Non-limiting examples of such methods include deletion of the CNTLN gene.

9p-arm loss can also be assessed by assessing 9p22.3 loss. Non-limiting assessment methods include assessment of deletion of one or more of the following genes: TTC39B; PSIP1; SNAPC3; or ZDHHC21.

9p-arm loss can be further assessed by 9p23 loss. Non-limiting assessment methods include assessing deletion of NFIB gene.

9p-arm loss can be further measured by assessing 9p24.1 loss which can be determined by assessing deletion of one or more or all of the following genes: KIAA2026; ERMP1; AK3; KDM4C; RANBP6; IL33; UHRF2; CDC37L1; RCL1; RLN2 or INSL6.

In addition prognostic prediction, the disclosed methods selectively predict immune checkpoint therapy (ICT) resistance, when the presence of 9p24.1 loss, 9p loss, and/or JAK2-CD274 co-deletion is detected. As used herein, co-deletion intends that both genes are deleted.

9p-arm loss can also be measured by assessing 9p24.2 loss. Non-limiting methods for determining this loss comprises deletion of one or more of the following genes: RFX3; SLC1A1 or VLDLR.

30) 9p-arm loss can further be measured by assessing 9p24.3 loss. Non-limiting methods for determining this loss comprises determining deletion of one or more of the following genes: SMARCA2; CBWD1 or KANK1.

In a yet further aspect, 9p-arm loss can further be measured by assessing 9p13.1 loss. Non-limiting examples for determining this comprises deletion of one or more of the following genes: CNTNAP3 and ZNF658.

In another embodiment, 9p-arm loss can further be measured by assessing 9p13.2 loss. Non-limiting examples to assess this loss comprises detecting or determining deletion or one or more of the following genes: ZBTB5; GRHPR; RNF38; FBXO10; DCAF1 O; ZCCHC7; EXOSC3; TOMM5; POLR1E; MELK; or SHB.

Assessing 9p13.3 loss is another embodiment to assess 9p-arm loss. Assessment of 9p13.3 loss comprises assessment of deletion of one or more of the following genes: NOL6; NFX1; BAG1; CHMP5; UBE2R2; UBAP2; UBAP1; DCAF12; UNC13B; CCDC107; PIGO; STOML2; VCP; TESK1; FANCG; DNAJB5; C9orf131; TLN1; GBA2; CREB3; CA9; RGP1; DCTN3; GALT; IL1 IRA; SIGMAR1; NUDT2; HINT2; or KIF24.

In a yet further embodiment, 9p-arm loss is assessed by measuring one or more of SASP, Cytokine-Cytokine-Receptor Interaction, JAK-STAT Signaling, and TNFA Signaling via NFkB. Alternatively or in addition, the method further comprises assessing a decrease in SASP, Cytokine-Cytokine-Receptor Interaction, JAK-STAT Signaling, and TNFA Signaling via NFkB.

9p-arm loss can also be assessed by measuring for a decrease in chemokines CXCL9 and CXCL10.

In any of the above methods, aspects, or embodiment, the method further comprises, consisting essentially of, or yet consists of measuring PD-L1 gene deletion and/or PD-L1 expression.

Yet further provided is a method for predicting or determining whether a subject with cancer will respond to treatment with immunotherapy, the method comprising, or consisting essentially of, or yet consisting of i) measuring 9p-arm loss in a cancer cell from the subject; ii) determining a degree of 9p-arm loss; and iii) determining that the subject will respond to administering PD-1/PD-L1 centered immunotherapy if the degree of 9p-arm loss does not show significant 9p-arm loss.

An additional disclosed method is for predicting or determining whether a type of cancer will respond to treatment with immunotherapy, the method comprising, or consisting essentially of, or yet further consisting of: i) measuring 9p-arm loss in a cancer cell from the subject; ii) determining a degree of 9p-arm loss; and iii) determining that the type of cancer will respond to administering PD-1/PD-L1 centered immunotherapy if the degree of 9p-arm loss does not show significant 9p-arm loss.

A method for treating cancer in a subject in need thereof is provided, the method comprising, or consisting essentially of, or consisting of administering PD-1/PD-L1 centered immunotherapy to the subject if a sample isolated from the patient comprising the cancer cell does not show 9p-arm loss.

An additional method for treating cancer in a subject in need thereof is provided, the method comprising, or consisting essentially of, or consisting of administering a therapy to the subject that does not comprise PD-1/PD-L1 centered immunotherapy to the subject if a sample isolated from the patient comprising the cancer cell shows 9p-arm loss.

Also provided is a method for treating cancer in a subject in need thereof, the method comprising, or consisting essentially of, or yet further consisting of measuring gene deletion of JAK2 and PD-L1 in a cancer cell from the subject; wherein when genetic studies of JAK2 and PD-L1 is not indicative of co-deletion of JAK2 and PD-L1, then the subject is administered PD-1/PD-L1 centered immunotherapy, and wherein when genomic assessment of JAK2 and PD-L1 is indicative of co-deletion of JAK2 and PD-L1, then the subject is not administered PD-1/PD-L1 centered immunotherapy, and an alternative treatment is administered to the subject.

Yet further provided is a method of inhibiting the growth or progression of cancer in a subject comprising, or consisting essentially of, or yet further consisting of i) measuring 9p-arm and 9p21 loss in a precancer cell from the subject; ii) determining a degree of 9p-arm loss; and iii) administering to the subject treatment that does not comprise PD-1/PD-L1 centered immunotherapy if the degree of 9p-arm loss shows significant 9p-arm loss. A precancer cell can be determined by a tissue biopsy.

When a patient is identified for treatment, various therapies are provided herein. When the patient is determined to benefit from PD-1/PD-L1 centered immunotherapy, a therapeutically effective amount of the therapy is administered. When the patient is determined not to benefit from CIT or e.g., PD-1/PD-L1 centered therapy, another therapy is administered. These can be administered as first-line, second-line, third-line, fourth-line, fifth-line therapy, and can be combined with tumor resection.

Thus, when the patient is determined to benefit from PD-1/PD-L1 centered immunotherapy, a therapeutically effective amount pembrolizumab or nivolumab is administered.

In addition, when the patient is determined not to benefit from PD-1/PD-L1, an alternative therapy is administered that does not comprise, or consist essentially of, or yet further consist of PD-1/PD-L1 centered immunotherapy if the degree of 9p-arm loss shows significant 9p-arm loss. Non-limiting examples of such include standard chemotherapy or cetuximab.

In some embodiments, the immunotherapy agent comprises, consists essentially of, or consists of one or more selected from monoclonal antibodies (such as a monospecific, bispecific or multispecific antibody recognizing a tumor-specific antigen and/or an immune checkpoint), antibody-drug conjugates (e.g., recognizing a tumor-specific antigen and/or an immune checkpoint wherein the conjugated drug kill or damage a cancer cell expressing the tumor-specific antigen and/or inhibit an inhibitory immune checkpoint and/or active a stimulating immune checkpoint), a CAR therapy, a cell therapy (e.g., transplanting an anti-cancer immune cell optionally amplified and/or activated in vivo, or administering an immune cell expressing a chimeric antigen receptor (CAR)), immune regulators, cancer vaccines, an inhibitor or antagonist of an inhibitory immune checkpoint (referred to herein as a “checkpoint inhibitor”, such as a chemical substance, an antisense oligonucleotide (ASO), a RNA interference (RNAi), a Clustered Regularly Interspaced Short Palindromic Repeat (CRISPR) system, a vector delivering each thereof), an activator or agonist of a stimulatory immune checkpoint (such as an activating ligand). In some embodiments, the immunotherapy agent comprises, consists essentially of, or consists of one or more monoclonal antibodies, bispecific antibodies and antibody fragments. In one embodiment, the immunotherapy agent comprises, consists essentially of, or consists of one or more of bispecific antibodies specifically binding to a tumor-specific antigen and engages an immune cell, such as a bispecific T-cell engager, a bispecific NK-cell engager, a bispecific NKT-cell engager, a bispecific gamma-delta T-cell engager, and a bispecific cytotoxic T-cell engager. In some embodiments, the immunotherapy agent comprises, consists essentially of, or consists of one or more antibody-drug conjugates. In some embodiments, the immunotherapy agent comprises, consists essentially of, or consists of one or more CAR cell therapy, such as administration of an immune cell expressing a CAR, including but not limited to CAR T cells, CAR NK cells, CAR NKT cells, CAR CD8+ T cells, CAR cytotoxic T cells, CAR gamma-delta T cells. In some embodiments, the immunotherapy agent comprises, consists essentially of, or consists of one or more cancer vaccines, such as a polypeptide or a polynucleotide mimicking a tumor-specific antigen and capable of inducing an immune response to the antigen in a subject. In some embodiments, the immunotherapy agent comprises, consists essentially of, or consists of one or more oncolytic virus therapy, such as a viral vector specifically infecting and optionally duplicating in a cancer cell and delivering an immunotherapy agent to the cancer cell. In one embodiment, the oncolytic virus is an HSV, optionally selected from HSV-1 and HSV-2. In a further embodiment, the oncolytic virus increases the expression optionally on the cell surface of a tumor-specific antigen in a cancer cell; and/or reduces the expression and/or activity of an inhibitory immune checkpoint in a cancer cell; and/or increases the expression and/or activity of a stimulatory immune checkpoint in a cancer cell.

Non-limiting examples of monoclonal antibodies include rituximab, blinatumomab, alemtuzumab, ibritumomab tiuxetan, bevacizumab, bevacizumab-awwb, cetuximab, panitumumab, ofatumumab, denosumab, pertuzumab, obinutuzumab, elotuzumab, ramucirumab, dinutuximab, daratumumab, trastuzumab, trastuzumab-dkst, nivolumab, pembrolizumab, cemiplimab, spartalizumab, camrelizumab, sintilimab, tislelizumab, toripalimab, AMF 514 (MEDI0680), balstilimab, avelumab, durvalumab, atezolizumab, ipilimumab, tremelimumab, zalifrelimab, and AGEN1181. In some embodiments, the monoclonal antibody is combined with another agent. For example, rituximab may be formulated with hyaluronidase human.

Non-limiting examples of antibody-drug conjugates include moxetumomab pasudotox-tdfk, brentuximab vedotin, trastuzumab emtansine, inotuzumab ozogamicin, gemtuzumab ozogamicin, tagraxofusp-erzs, polatuzumab vedotin-piiq, enfortumab vedotin-ejfv, trastuzumab deruxtecan, and sacituzumab govitecan-hziy.

Non-limiting examples of CAR T-cell therapy include tisagenlecleucel and axicabtagene ciloleucel.

Non-limiting examples of immune regulators include interleukins, aldesleukin, interferon alfa-2a/2b, pexidartinib, erythropoietin, granulocyte-macrophage colony-stimulating factor (GM-CSF), granulocyte colony-stimulating factor (G-CSF), thalidomide, lenalidomide, pomalidomide, and imiquimod.

Non-limiting examples of cancer vaccines include BCG live (THERACYS®) or sipuleucel-T (PROVENGE®).

Non-limiting examples of oncolytic virus therapy include oncorine (H101) and talimogene laherparepvec (IMLYGIC®).

In some embodiments, the immunotherapy agent comprises, consists essentially of, or consists of a checkpoint inhibitor.

In some embodiments, the checkpoint inhibitor comprises, consists essentially of, or consists of a non-antibody agent. In some embodiments, the checkpoint inhibitor comprises, consists essentially of, or consists of GS4224, AMP-224, CA-327, CA-170, BMS-1001, BMS-1166, peptide-57, M7824, MGD013, CX-072, UNP-12, NP-12, or a combination of two or more thereof.

In some embodiments, the checkpoint inhibitor comprises, consists essentially of, or consists of one or more selected from an anti-PD-1 agent, an anti-PD-L1 agent, an anti-CTLA-4 agent, an anti-LAG-3 agent, an anti-TIM-3 agent, an anti-TIGIT agent, an anti-VISTA agent, an anti-B7-H3 agent, an anti-BTLA agent, an anti-ICOS agent, an anti-GITR agent, an anti-4-1BB agent, an anti-OX40 agent, an anti-CD27 agent, an anti-CD28 agent, an anti-CD40 agent, and an anti-Siglec-15 agent. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an antagonist. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an agonist. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an inhibitor. In some embodiments, the anti-LAG-3 agent comprises, consists essentially of, or consists of AK104, KN046, eftilagimod alpha, relatlimab, LAG525, MK-4280, REGN3767, TSR-033, BI754111, Sym022, FS118, or MGD013. In some embodiments, the anti-TIM-3 agent comprises, consists essentially of, or consists of CA-327, TSR-022, MBG453, Sym023, INCAGN2390, LY3321367, BMS-986258, SHR-1702, or R07121661. In some embodiments, the anti-TIGIT agent comprises, consists essentially of, or consists of MK-7684, etigilimab, tiragolumab, BMS-986207, AB-154, or ASP-8374. In some embodiments, the anti-VISTA agent comprises, consists essentially of, or consists of JNJ-61610588 or CA-170. In some embodiments, the anti-B7-H3 agent comprises, consists essentially of, or consists of enoblituzumab, MGD009, or omburtamab. In some embodiments, the anti-BTLA agent comprises, consists essentially of, or consists of TAB004/JS004. In some embodiments, the anti-Siglec-15 agent comprises, consists essentially of, or consists of NC318. In some embodiments, the checkpoint inhibitor comprises, consists essentially of, or consists of AK104 or KN046.

In some embodiments, the checkpoint inhibitor comprises, consists essentially of, or consists of an anti-PD1 agent or an anti-PD-L1 agent.

In some embodiments, the anti-PD1 agent comprises, consists essentially of, or consists of an anti-PD1 antibody or an antigen binding fragment thereof. In some embodiments, the anti-PD1 antibody comprises, consists essentially of, or consists of nivolumab, pembrolizumab, cemiplimab, spartalizumab, camrelizumab, sintilimab, tislelizumab, toripalimab, AMF 514 (MEDI0680), balstilimab, or a combination of two or more thereof.

In some embodiments, the anti-PD-L1 agent comprises, consists essentially of, or consists of an anti-PD-L1 antibody or an antigen binding fragment thereof. In some embodiments, the anti-PD-L1 antibody comprises, consists essentially of, or consists of avelumab, durvalumab, atezolizumab, envafolimab, or a combination of two or more thereof.

In some embodiments, the checkpoint inhibitor comprises, consists essentially of, or consists of an anti-CTLA-4 agent. In some embodiments, the anti-CTLA-4 agent comprises, consists essentially of, or consists of an anti-CTLA-4 antibody or an antigen binding fragment thereof. In some embodiments, the anti-CTLA-4 antibody comprises, consists essentially of, or consists of ipilimumab, tremelimumab, zalifrelimab, or AGEN1181, or a combination thereof.

Cancers suitably treated by the disclosed methods are discussed above and incorporated herein. In one aspect, the cancer comprises a solid tumor, e.g., melanoma, esophageal cancer, non-small cell lung cancer [both lung adenocarcinoma and lung squamous cell carcinoma], pancreatic cancer, gastric [stomach] cancer, bladder cancer, head and neck cancer or an oral cavity cancer. The cancer can be Stage I, Stage II, Stage III or Stage IV. The therapy can be first-line, second-line, third-line, fourth line or fifth line. In aspect, the cancer or precancer is not renal cancer.

In some embodiments, the cancer cell or precancer cell is a primary cell isolated from a biopsy. Alternatively when the method is used in vitro, the cancer or precancer cell can be a primary cell or a cultured cancer cell that is cultured in the lab or obtained from a commercial vendor such as the American Type Culture Collection (ATCC), or a cancer cell in an animal model for evaluating therapeutic efficacy of potential therapies.

Administration and Dosing

The appropriate amount and dosing regimen of the active agent to be administered to the subject according to any of the methods disclosed herein, is determined by one of ordinary skill in the art. In some embodiments, the active agents, or salts or solvates thereof, is administered to a subject suffering from abnormal cell growth, such as a human, either alone or as part of a pharmaceutically acceptable formulation, once a week, once a day, twice a day, three times a day, or four times a day, or even more frequently.

Administration can be effected by any method that enables delivery of the compounds to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the chemotherapeutic or immunotherapeutic agent are well-known in the relevant art and would be understood to be encompassed by the skilled artisan once provided the teachings disclosed herein.

In some embodiments, nivolumab is administered as a dose of 240 mg once every 2 weeks. In some embodiments, nivolumab is administered as a dose of 480 mg once every 4 weeks.

The following examples are included to demonstrate some embodiments of the disclosure. However, those of skill in the art should, in light of the present disclosure, appreciate that many changes can be made in the specific embodiments which are disclosed and still obtain a like or similar result without departing from the spirit and scope of the invention.

Experimental Methods

An aneuploid-immune paradox encompasses somatic copy-number alterations (SCNAs) unleashing a cytotoxic response in experimental precancer systems, while conversely being associated with immune suppression and cytotoxic-cell depletion in human tumors, especially head and neck cancer (HNSC). Applicant report here that alterations in chromosome dosage contribute to an immune hot-to-cold switch during human HPV-negative head and neck tumorigenesis. Copy-number correlates of immune and neoplastic transition were characterized in HPV-negative HNSC (two patient cohorts), its precursor in a large prospective cohort and cell line panel. Overall SCNA level was associated with increased CD3+ and CD8+ T-cell microenvironments in precancer (mostly linked to trisomy and tetrasomy), but with T-cell deficient tumors. Early lesions with 9p21.3 loss and high SCNA levels were associated with depletion of cytotoxic T-cell- (especially in TP53 mutant tumors) and NK-cell infiltration, respectively. The strongest predictor of T-cell depletion in oral cancer was 9p loss, reflecting cumulative gene-dosage decreases across the arm. 9p21.3 deletion contributed mainly to cell-intrinsic senescence suppression, but deletion of the entire 9p arm was necessary to diminish levels of cytokine, JAK-STAT, and Hallmark NFkB pathways. Finally, 9p arm-level loss and JAK2-PD-L1 co-deletion (at 9p24) were predictive markers of poor survival in recurrent HPV-negative HNSC after anti-PD-1 therapy. Applicant hypothesize that 9p21.3 arm-loss expansion, to incorporate neighboring cooperating, regulatory genes, allows oral precancer cells to acquire properties to overcome a pro-immunogenic aneuploid checkpoint and escape immune attack. These findings enable distinct HNSC interception and precision-therapeutic approaches, concepts that may apply to other neoplastic sites, aneuploid diseases, and immunotherapy.

### Prospective Oral Precancer Cohort

Clinical, demographic and genomic studies. The prospective cohort included 188 oral precancer patients, characterized by clinical, pathologic and SCNA risk factors. After study enrollment, patients were stratified by prior oral cancer, and systematically followed until reaching the protocol-specified primary endpoint of invasive cancer. Patients had clinic visits at months 1, 3, 6, 9, 12, and every 6 months thereafter following protocol-defined and institutional practice. Additionally, an oral cancer-free survival (OCFS) sweep was performed prior to database lock for the current analysis. The protocol was registered in ClinicalTrials.gov (NCT00402779) and approved by the MD Anderson Cancer Center Institutional Review Board. Participants provided written informed consent for biospecimens to be tested for genomic alterations and status of other biomarkers of interest reported herein, as well as for collection of demographic, clinical and outcomes data. Precancer SCNAs included previously established major (3p14, 9p21.3, and 17p13.1) and minor (4q26-28, 4q31.1, 8p22, 8p23, 11q13, 11q22, and 13q21) CN-loss risk loci (13, 78) and chromosome 7 gain. See SI Appendix for additional Methods details, including SCNA, Genomic and Immune Profiling, and Statistical Considerations for this and the other major sections below.

### HPV-Negative HNSC in the Cancer Genome Atlas (TCGA)

SCNA, mutation, gene expression, HPV status and clinical parameters. CN data of HNSCs in TCGA cohort were derived from Affymetrix SNP 6.0 arrays and obtained from the GDAC Firehose and GDC Data Portal (GISTIC2 analysis, Level4). The copy numbers for each chromosomal region (given as log 2 CN ratios) were adjusted by tumor purity derived from previously reported pathology-based and ABSOLUTE (23, 24, 26, 79-81) methods. The HNSC SCNA level corresponds to the total number of chromosome arm gains or losses across the genome (except where otherwise specified). To distinguish between arm and focal-level events, Applicant considered a threshold of ≥70% (default value in GISTIC2) of arm length (given in units of the fraction of chromosome arm) to identify the arm-level events, while all the others were considered as focal-level events. All the multivariable regression analyses that included a specific SCNA (e.g., 9p21.3 loss) as binary (loss or no loss) were confirmed using log 2-transformed CN ratio as continuous variable (after normalized to z-score (23)). HPV-positive HNSC was based on rigorous viral-read (27) and anatomic subsite data criteria.

### HPV-Negative HNSC Cell Lines

SCNA and gene expression data. Applicant studied 32 HPV-negative HNSC cell lines (SCC9, SCC25, DETROIT562, BICR31, SCC4, SCC15, BICR6, HSC2, SNU46, BICR16, CAL33, HSC4, BHY, SNU1076, PECAPJ34CLONEC12, SNU1041, PECAPJ15, YD8, SNU1066, SNU899, SNU1214, YD10B, PECAPJ41CLONED2, PECAPJ49, A253, YD38, BICR56, HSC3, CAL27, YD15, FADU, BICR18). Segments, SCNA, and gene-expression data were derived from DepMap (CCLE_segmented_cn.csv, DepMap Public 19Q4; CCLE_gene_cn.csv, DepMap Public 19Q4 and CCLE_expression_full.csv, DepMap Public 19Q4), respectively (82).

### Survival Analysis of 9p Loss in ICB Treated HPV-Negative HNSC

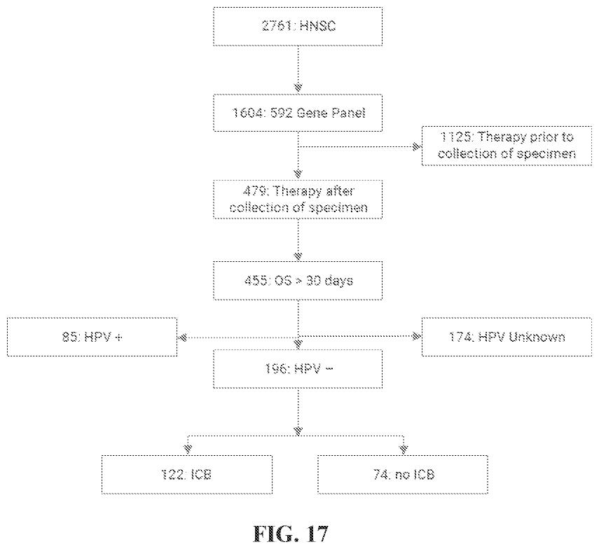

Real-World Evidence (RWE) cohort. Applicant utilized a real-world cohort of HPV-negative HNSC patients treated with PD-1 inhibitors nivolumab or pembrolizumab, or chemotherapy, whose tumors were microdissected then underwent next-generation sequencing of a panel of 592 genes covering all autosomes and the X chromosome. Applicant used this information to infer CN (loss) at 3p, 9p, and 17p (Consort diagram, FIG. 17). The 592-gene panel arm loss algorithm was CAP/CLIA validated against: standard FISH in 436 patients for 1p/19q co-deletion (24) showing sensitivity of 96.6% (95% CI: 82.2-99.9) and specificity of 99.5% (95% CI: 98.2-99.9); further validated against whole exome sequencing on 369 cases for 9p and 9p21 loss, finding a Pearson's correlation between the two assays of 0.828 for 9p loss and 0.773 for 9p21.3 loss. TMB in Applicant's 592-gene panel was found to be equivalent to the FDA-approved 324-gene companion diagnostic test related to the agnostic use of pembrolizumab for tumors with ≥10 mutations/megabase. The 592-gene MI Tumor Seek panel was used to validate anti-PD-1 therapy efficacy in relation to MSI status (see Supplemental Materials).

### Prospective Oral Precancer Cohort

SCNA and Multiplex immune profiling. Chromosome gain was determined by fluorescence in situ hybridization, using a chromosome-7 centromeric probe, randomly selected marker of tri-/tetra-somy based on similar phenotypes with different chromosomes in this context (W. N. Hottelman, Ann N Y Acad Sci 952, 1-12 (2001)). After specific PCR-DNA amplification, an automatic capillary DNA analyzer was used to separate microsatellite alleles and to quantify the peak height of individual alleles for each marker. Loss of at least one of the markers (using the criteria described in the statistical section) was considered as loss of that specific chromosomal site. Applicant considered overall SCNA level as chr7 gain and loss at 7 chromosome arms, including 3p, 9p, and 17p and the following: 4q, 8p, 11q, and 13q.

As described in a prior methods report (E. R. Parra et al., Scientific Reports 7, 13380 (2017)), Applicant utilized a validated multiplex immunofluorescence (mIF) panel of five antibodies stained on the same tissue section, and labeled using a tyramide-signal amplification-based kit, including: anti-CD3 (Dako, T lymphocyte marker), anti-CD8 (clone C8/144B, Thermo Scientific, present on cytotoxic T cells), anti-CD68 (clone PG-M1, Dako, macrophage lineage marker), anti-cytokeratin (clone AE1/AE3, Dako), and DAPI (nuclear staining). Each antibody was labeled with a specific fluorophore. All antibodies had been optimized for mIF by examination of positive and negative controls and testing of the antibodies by Western blotting. Applicant performed scanning and image capture with a multispectral microscope (Vectra™, PerkinElmer), and analyzed the images with a specialized software (InForm™, PerkinElmer) capable of counting the number of cells with positive staining for each marker in a specified area. For each sample, 1-10 representative areas (median 6) measuring 1 mm2 each, were randomly selected for marker quantification. CD3+ and CD8+ T-cells and CD68+ macrophages were evaluated and reported as cell density (i.e., cells/mm2).

Statistical considerations. Wilcoxon rank-sum tests were used to compare the distribution of continuous variables between two groups defined by a binary variable. Fisher's exact tests were used to assess the association between binary markers and categorical factors. Spearman correlation coefficients were used to evaluate pair-wise correlations between two markers in continuous scale. When analyzing biomarkers assessed in a continuous scale, values from multiple areas were transformed into logarithm scale and were fit with linear mixed-effect models to account for within-patient variation (J. Jiang Springer Series in Statistics (Springer-Verlag New York, ed. 1, 2007)). OCFS (model1) was defined as the time from protocol registration until development of the protocol-specified primary endpoint of invasive oral cancer, or death, and estimated by the Kaplan-Meier method. Cox proportional hazard model was used to study marker and OCFS associations. Log-rank test was performed to test the difference in OCFS between groups. All analyses were performed using SAS software (version 9.4; SAS Institute, Cary, NC) and R.

Cox Model˜9p21.3Loss+3p14Loss+17p13.1Loss+Histology(model1)

Throughout the paper, in the univariate (model2) and multivariable (model3) analyses performed to predict the level of a specific parameter (e.g., immune-cell level) based copy-number event, two different cutoffs were used (35%-65%, and confirmed with a 50%). For example, in multivariable logistic regression of precancer cases, Applicant divided the patients into the top and bottom 35% in terms of percent of immune cells and used loss at 3p14, 9p21.3, or/and 17p13.1, and SCNA level, to determine the contribution of each parameter to the immune-cell landscape. The same cutoffs were used for the analysis of TCGA cases, including IS. The chromosome loss in precancer is defined as the ratio of the peak heights of the two alleles in lesion (L1/L2) DNA and in the corresponding normal lymphocytes (N1/N2) DNA>1.43 or <0.7, consistent with the same cutoffs utilized in the landmark clinical study that prospectively correlated precancer-specific SCNAs with prognosis (W. N. William, Jr. et al., JAMA oncology 2, 209-216 (2016)).

The SCNA level was calculated based on the available information on the SCNA level at different genomic loci. More specifically, information was available on the presence of losses at the following loci: 3p21-14, 4q26-28, 4q31.1, 8p22-p23, 9p21.3, 11q13-q22, 13q21, 17p13.1—SCNA level was calculated based on any SCNA (loss at all microsatellite loci in 3p21-14, 4q26-28, 4q31.1, 8p22, 8p23, 9p21.3, 11q13, 11q22, 13q21, or 17p13.1 and/or chromosome 7 gain):

$$SCNA . {level}^{*} = ( \begin{matrix}no SCNA & 0 ( none ) \\ any Loss OR chr 7 gain & 1 ( low ) \\ any Loss AND chr 7 gain & 2 ( high )\end{matrix} )$$

$${Immune}_{infiltrate} \sim 3 p 14 Loss or 9 p 21.3 Loss or 17 p 13.1 Loss \times β + ε ( random effect ) \qquad ( model 2 )$$

$${Immune}_{infiltrate} \sim 3 p 14 Loss + 9 p 21.3 Loss + 17 p 13.1 Loss + SCNA . level \qquad ( model 3 )$$

### HPV-Negative HNSC in the Cancer Genome Atlas (TCGA)

Statistical considerations. Logistic regression was used to determine the variables most predictive of different parameters representing immune infiltrate (model4-5) and IS levels. All the findings were confirmed using two purity methods (pathology-based and ABSOLUTE, see main manuscript Methods) and reported in the supplemental tables. After purity adjustment, Applicant consider a log 2-transformed copy number ratio>0.3 as a gain and <(−0.3) as a loss. Gene expression, mutation and clinical data were downloaded from GDAC Firehose. Patients with nonsense, splice site, frame shift, and missense mutations with Polyphen2-HVAR score>0.2 (I. A. Adzhubei et al., Nat Methods 7, 248-249 (2010)) were considered as TP53 mutation-positive patients. HPV-status was determined using a cutoff of RPM>1 (viral reads per million) in tumors of oropharyngeal origin was used as a stringent definition of HPV-positive HNSC, for a limited subset analysis. Applicant used the log 2 transformed RSEM values to estimate differences in the immune cell content (different types of immune cells) using CIBERSORT (using the LM22 gene signature) (A. M. Newman et al., Nat Methods 12, 453-457 (2015)). In order to recapitulate in HNSC what was done in oral precancers, RNA expression levels of specific immune markers were applied as a proxy of the level of the corresponding immune cells in the tumor. Previous studies have shown that RNA expression levels (e.g., CD8+) of immune-cell markers highly correlate with cell estimates based on immunofluorescence (J. E. Beane et al., Nat Commun 10, 1856 (2019); A. Mezheyeuski et al., J Pathol 244, 421-431 (2018)) (see Results). Applicant defined the tumors as having low or high gene expression level using the 35th and 65th percentiles; results were confirmed using a cutoff of 50%. Applicant used the GISTIC2 algorithm to distinguish between the two major types of events—arm-level and focal-level events. When specified, logistic regression was performed after first selecting patients with a certain tumor stage or containing or not TP53 mutations. In addition, 5-fold cross-validation and variable-importance (size-effect) evaluation (package caret) were also performed to evaluate the accuracy of multivariable logistic regression models, and the size effect of each covariate, respectively.

$$SCNA level = \sum arm gains + \sum arm losses$$

$${Immune}_{infiltrate} \sim 3 p 14 Loss + 9 p 21.3 Loss + 17 p 13.1 Loss + Chr 7 Gain + SCNA level ( normalized ) \qquad ( model 4 )$$

$${Immune}_{infiltrate} \sim 9 p 21.3 Loss ( arm ) + 9 p 21.3 Loss ( focal ) + 9 p 21.3 Loss ( arm + focal ) + 3 p 14 Loss + SCNA level ( normalized ) \qquad ( model 5 )$$

For the least absolute shrinkage and selection operator (Lasso) (R. Tibshirani, Journal of the Royal Statistical Society. Series B (Methodological) 58, 267-288 (1996)) classification, to determine the parameters most predictive of immune-infiltrate level, Applicant defined tumors as having low or high IS levels using the 35th and 65th percentiles, as described previously (T. Davoli et al., Science 355 (2017)), and used a binomial model. Applicant also applied Lasso using 10-fold cross validation. As variables, Applicant included the presence of loss or gain in all possible whole chromosome or chromosome arms across the genome and the overall arm/chromosome SCNA level.

For GSEA (Gene Set Enrichment Analysis) comparing tumor samples with or without the indicated SCNA, Applicant first used DEseq2 package (M. I. Love et al., Genome Biol 15, 550 (2014)) to calculate the log 2 fold change and adjusted p-value of differentially expressed genes. Then Applicant calculated the differential-expression score [Exp. Score] based on the formula below. Exp. Score was then used to perform GSEA pathway analysis (pre-ranked model) (12).

Exp. Score=sign(log₂ FoldChange)×−log₁₀(adjusted p_value)

### HPV-Negative HNSC Cell Lines

Statistical considerations. Applicant used GSEA to investigate mechanisms of 9p21.3 or 3p14 (selected based on statistical significance in TCGA analyses) effects on gene- and pathway-change. To have similar numbers of cells in each group Applicant split the cell lines into those having a copy-number level lower or higher than the median value (for example, as a threshold Applicant used the median based on log 2 copy-number ratio of −0.45 for 9p21.3 and −0.5 for 3p14 region). Applicant used DEseq2 (M. I. Love et al., Genome Biol 15, 550 (2014)) to estimate the differential expression between the two groups of cell lines. Applicant then used the formula above to calculate a score as input in GSEA (A. Subramanian et al., Proceedings of the National Academy of Sciences of the United States of America 102, 15545-15550 (2005)). For GSEA, Applicant used the comprehensive list of pathway and gene sets from BIOCARTA, KEGG and REACTOME databases. Applicant performed logistic regression (model6) to determine the parameters most predictive of the expression of the SASP signature pathway (see below). Applicant distinguished cell lines as having low or high SASP-signature scores using the 35th/65th, or 50th percentile cutoff (binary) or continuous variable of the distribution (all the continuous variables were normalized before being used in the multivariable models). Applicant applied a logistic model on the dataset using as variables, 3p14 loss or 9p21.3 loss. To determine the contribution of chromosome loss encompassing whole 9p, 3p, and 17p arms as compared to focal losses, Applicant used GISTIC2. Applying GISTIC2 algorithm to segment data derived from the cell lines, Applicant determined the type of loss at 9p21.3 and 3p14: arm-level event versus focal-only event. As in TCGA analyses, a threshold of 70% of arm length (given in units of the fraction of chromosome arm) was used in GISTIC2 to distinguish between cell-line focal- and arm-level events.

SASP enrichment˜9p21.3Loss(arm)+9p21.3Loss(focal)(model6)

SASP and IFNα gene set. To derive an aneuploidy-associated SASP signature, Applicant first considered the genes upregulated in SASP as previously reported (J. P. Coppe et al., PLoS Biol 6, 2853-2868 (2008)). Then Applicant crossed this gene list with the genes upregulated (log 2FC of at least 1.5) in aneuploid cells versus control cells (reversine treated versus control) from (S. Santaguida et al., Genes Dev 29, 2010-2021 (2015)). The derived list of 20 genes represents the SASP signature (underlying data not shown). The IFNα gene set contains the list of IFNα genes that are located on chromosome 9p21.3 (data not shown). To calculate the SASP gene-expression signature and IFNα gene expression Applicant used the single-sample GSEA (ssGSEA package) (A. Subramanian et al., Proceedings of the National Academy of Sciences of the United States of America 102, 15545-15550 (2005)). Survival analysis of 9p loss in ICB treated HPV-negative HNSC

Statistical, profiling considerations for real-world evidence (RWE) cohort. Applicant employed a novel “virtual karyotyping” platform that allowed interrogation of the associations between loss of genes/chromosomal regions in 9p or 3p and outcomes using linked biomarker-EMR-insurance claim-survival record data. The Caris Life Sciences CODEai database which contains over 215,000 molecular profiles combined with clinical outcomes was leveraged for this study. 2,761 HNSC cases were available of which 1,604 had results from the Caris 592-gene assay. These cases were then segregated by whether therapy was administered prior to the collection of the sample that was profiled. 479 cases were found to have therapy administration only after the collection date of the tumor profiled. The 479 cases were further subdivided into groups based on whether immunotherapy (nivolumab or pembrolizumab) was part of their treatment. An overall survival minimum of 30 days was selected to remove patients with incomplete outcomes records (filtered down to 455). 196 of the 455 cases were HPV negative assessed by p16 IHC status. 122 patients were treated with immunotherapy across various lines of treatments with and without chemotherapy. 74 patients were found to have no immunotherapy treatment (Consort diagram, FIG. 17). TMB in Applicant's 592-gene panel was found to be equivalent to the FDA-approved 324-gene companion diagnostic test related to the agnostic use of pembrolizumab for tumors with ≥10 mutations/megabase (D. M. Merino et al., J. Immunother Cancer 8 (2020)). The 592-gene MI Tumor Seek panel was used to validate anti-PD-1 therapy efficacy in relation to MSI status (D. T. Le et al., Science 357, 409-413 (2017)).

Microdissection was performed on all cases and only those that achieved a minimum of 20% of tumor content in the area selected for microdissection were sequenced. Applicant derived the SCNA level by comparing the depth of sequencing of genomic loci to a diploid control as well as the known performance of these genomic loci from a CLIA-validated commercially available assay (MI Tumor Seek 592-gene panel; Caris Life Sciences), as recently described (K. Zimmer et al., Cancers (Basel) 12 (2020)). The 592-gene panel arm loss algorithm was CAP/CLIA validated in a study comparing 436 patients with both 592-gene panel arm-loss predictions and FISH results for 1p/19q co-deletion, a pattern shown to be therapeutically relevant in glial-brain tumors, e.g., low-grade gliomas (A. M. Taylor et al., Cancer Cell 33, 676-689 e673 (2018)). There were 29 samples positive by FISH and 407 samples negative by FISH for 1p/19q co-deletion and the arm-loss algorithm predictions had a sensitivity of 96.6% (95% CI: 82.2-99.9) and specificity of 99.5% (95% CI: 98.2-99.9). The 592-gene panel arm loss algorithm was further validated against the Caris Life Sciences Whole Exome Sequencing (WES) assay on 369 cases with both 592-gene panel arm-loss predictions and WES arm-loss predictions. WES used a conservative copy-number estimate across entire chromosomes, excluding centromeres and telomeres. This estimate included ˜50,000 intronic and intergenic SNPs which were present at minimum every 160,000 base intervals (6.25/megabase) along with depth data from every gene. The calculation used CNVKit (python 3.7, version 0.9.6) and provided confidence in deletion status by examining tens of thousands of data points and returning a call based on consecutive measurements of presence or absence of DNA. The Pearson's correlation between the two assays was 0.828 across 9p loss and 0.773 for 9p21.3 loss. PD-L1, PD-L2, and JAK2 were evaluated for copy-number loss using 1.6 copies or less to determine if the gene is deleted as described previously (J. P. Abraham et al., Clinical Cancer Research 10.1158/1078-0432.Ccr-20-3286, clincanres. 3286. 2020 (2020)). The 592-gene panel arm-loss algorithm evaluated large-scale gene deletions across both arms of all chromosomes where a gene was profiled. The mean copy number across all genes profiled on a region or arm was compared to the mean of all copies for genes profiled on the opposite arm. The resulting ratios for each chromosome must be ≤0.8 and the opposite arm cannot have an average copy number greater than 2.5 for the region or arm under consideration to be evaluated as lost. Larger cohorts using whole-exome or whole genome sequencing data will be important to extend these findings, including to address the contribution of TP53 mutations to ICB resistance, an analysis currently limited by statistical power due to the high TP53 mutation-9p loss co-occurrence rates.

### Results

### Oral Precancer Prospective Cohort

A clinical cohort of 188 HPV-negative oral precancer patients (median age 56 years old, range 23-82 years) was assembled who were systematically followed and rigorously annotated until reaching the protocol-specified primary endpoint of invasive cancer. The 5-year oral cancer-free survival (OCFS) for this population was 71.8%, with a median follow-up time for censored observations of 90.8 months. Multiplex immunofluorescence was used to assess the amount of infiltrating CD3+, CD8+, and CD68+ cells. This investigation was focused on the association of immune-cell infiltrate with three SCNA metrics: 1) reported major oral-cancer risk SCNAs, CN loss at 3p14, 9p21, and/or 17p13 (T. Davoli et al., Science 355 (2017); J. P. Coppe et al., PLoS Biol 6, 2853-2868 (2008); D. M. Merino et al., J Immunother Cancer 8 (2020)), 2) chromosome-7 gain (extra copies of this, or other single-chromosome gains, have similar precancer phenotypes, and are thus used to mark lesion trisomy or tetrasomy (M. I. Love et al., Genome Biol 15, 550 (2014))), and J. Jiang, Springer Series in Statistics (Springer-Verlag New York, ed. 1, 2007), 10.1007/978-0-387-47946-0, pp. 257) overall SCNA, or aneuploidy, level. The latter comprised all CN events, including CN loss at major and minor risk regions on six chromosome arms plus chr7 gain, in Applicant's precancer molecular profiling platform (see Methods). CN loss at 3p14, 9p21, or 17p13, or chromosome-7 gain tended to co-occur, particularly 3p14 loss and 9p21 loss with a p=1.29E-07 (event frequency and co-occurrence rates; data not shown).

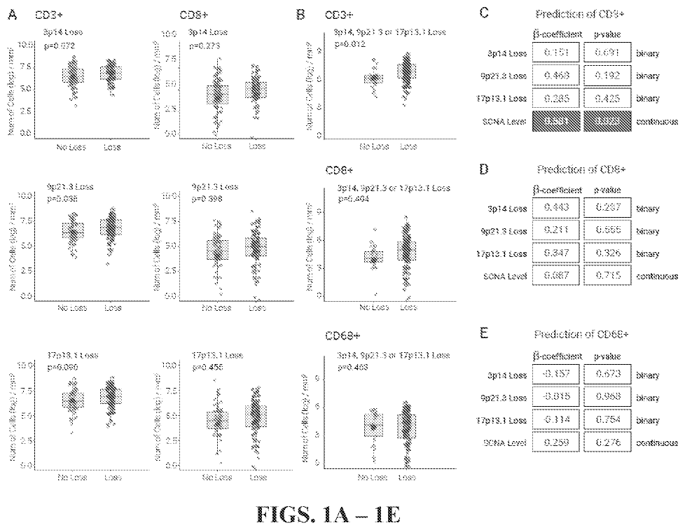

In precancer, SCNA level, especially chromosome trisomy/tetrasomy, was associated with increased CD3+ and CD8+ T-cell infiltrates. Applicant first correlated major-risk SCNAs (i.e., 3p14, 9p21, and/or 17p13 loss) with immune-cell infiltrate (FIG. 1). When considered individually (in univariate analysis), 9p21.3 loss was associated with a near two-fold increase in CD3+ cell density (p=0.038) (FIG. 1A); 3p14 loss (p=0.072) and 17p13.1 loss (p=0.090) were associated with increased CD3+ cell trends. Overall SCNA level was associated with increased CD3+(p=0.023), but not CD8+(p=not significant), cell infiltrate. Chr7 trisomy and tetrasomy was strongly associated with immune-hot precancer lesions—CD3+(p=0.0004) and CD8+(p=0.015) T-cell infiltration in univariate, 35% mixed-effect models (confirmed with median, 50%, cut off). Since SCNAs tended to co-occur (data not shown), multivariable logistic regression was performed to assess the contribution of each individual CN alteration and overall SCNA level to cell infiltrates, independent of the others (FIGS. 1C-1E). Chromosome gain (trisomy, tetrasomy) was the dominant driver of immune-cell infiltration in precancer lesions in multivariable analysis (CD3+, p=0.008; CD8+, p=0.058; CD68, p=0.051); contributing to the overall SCNA level-hot precancer association. When chr7 gain or SCNA level were taken into account, 3p14, 9p21.3, and/or 17p13.1 did not correlate with CD3+, CD8+, or CD68+ cell levels (FIGS. 1C-1E). In every precancer analysis—regardless of CN or immune metric, cut off (and whether univariate or multivariate)—SCNAs were never associated with a decreased T-cell count in the microenvironment.

9p21.3 loss (followed by dysplasia) was the strongest, independent prognostic marker of poor OCFS. The presence of dysplasia in oral precancer has been previously associated with cancer risk (6-8). As expected (12), dysplastic lesions harbored more SCNAs overall and 9p21 loss. A disproportionately stronger SCNA-dysplasia association was observed with CD3+ (vs. CD8+) cell levels, possibly reflecting increased CD3/CD4+ T-regs in histologic high-grade lesions (FIGS. 1A-1D). Compared to non-dysplastic lesions, dysplasia was associated with an increase in CD3+ cells (p<0.01) (data not shown). To determine the contributions of each clinical, histological, genomic, and immune marker in predicting outcomes, a univariate analysis of OCFS was performed. Applicant's parameters predicted poor OCFS: histology (dysplasia versus hyperplasia; β=0.693, p=0.009, HR=2.000, 95% CI=1.180-3.370), 9p21.3 loss (β=0.659, p=0.011, HR=1.932, 95% CI=1.160-3.200), chr7 gain (β=0.601, p=0.010, HR=1.824, 95% CI=1.151-2.889), and overall SCNA level (β=0.372, p=0.010, HR=1.451, 95% CI=1.092-1.927) (data not shown).

Next, a multivariable Cox-proportional hazard model including histology, three major risk SCNAs (loss at 3p14, 9p21, and/or 17p13), chr7 gain and overall SCNA was built, and demonstrated that only dysplasia and 9p21 loss were individually associated with poor OCFS (FIG. 6). Patients with both dysplasia and 9p21.3 loss had the lowest OCFS (p=0.0001; FIG. 6B); statistically significantly lower OCFS than patients with dysplasia or 9p21-loss alone (p=0.014 or p=0.012, log-rank test, respectively). Including age, gender, smoking status, alcohol use, T-cell infiltrates, major CN-loss loci, overall SCNA level, or chr7 gain in eight distinct multivariable prediction models revealed 9p21 loss as having the most consistent, significant, negative association with OCFS, followed by, and independent of, histology (FIG. 6C).

### Oral Cancer—HPV-Negative HNSC (TCGA)

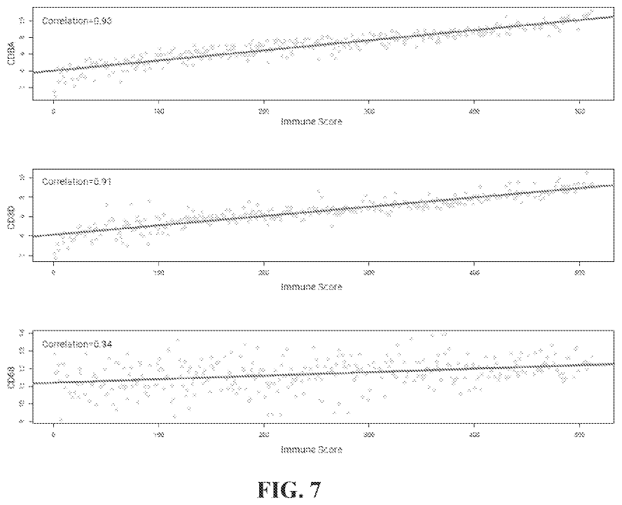

In TCGA HPV-negative HNSC, SCNAs were associated with decreased T-cell infiltrate: pivotal role of 9p loss. The comprehensive genomic and transcriptomic data from TCGA was leveraged to investigate in 343 HPV-negative HNSC samples the associations of 3p14, 9p21, and/or 17p13 loss and SCNA level with tumor-cell infiltrates and the previously described Immune Score (IS) (23). The IS was based on the expression level of cytotoxic T-cell markers, consistent with metrics utilized in other reports (24-29). RNA expression levels of CD3D, CD8A, or CD68 was used as a proxy to recapitulate in HNSC; the analyses performed on oral precancer biopsy samples for CD3+, CD8+, and CD68+ levels. Expression levels of CD3+ and CD8+, but not CD68+ (Pearson's r=0.34), strongly correlated (Pearson's r=0.91 and 0.93 for CD3D and CD8A, respectively) with IS (FIG. 7).

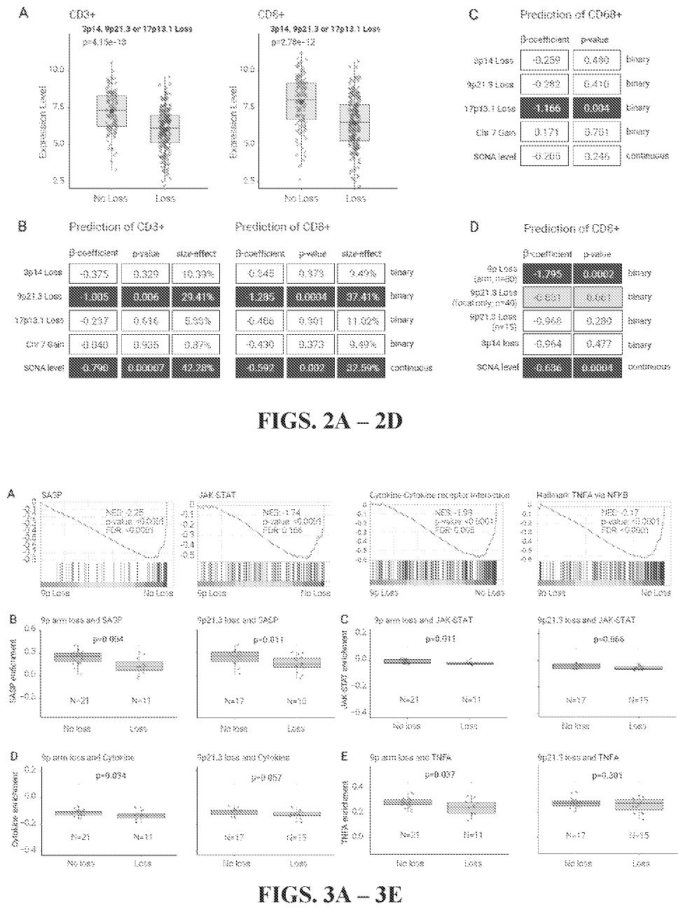

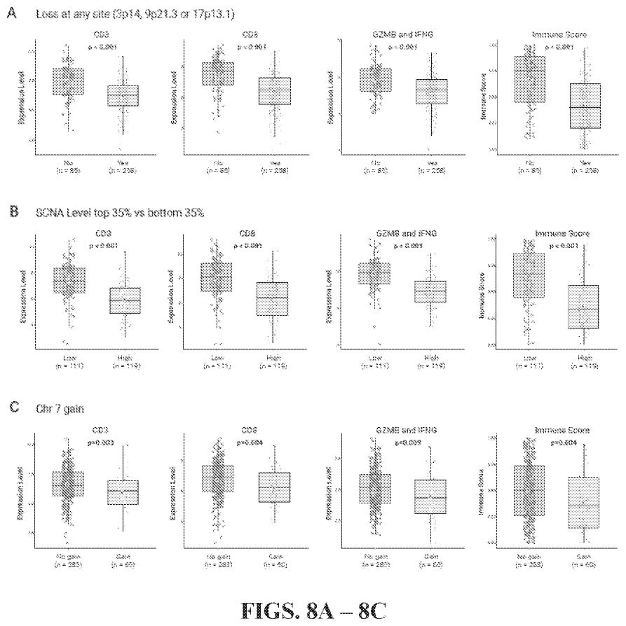

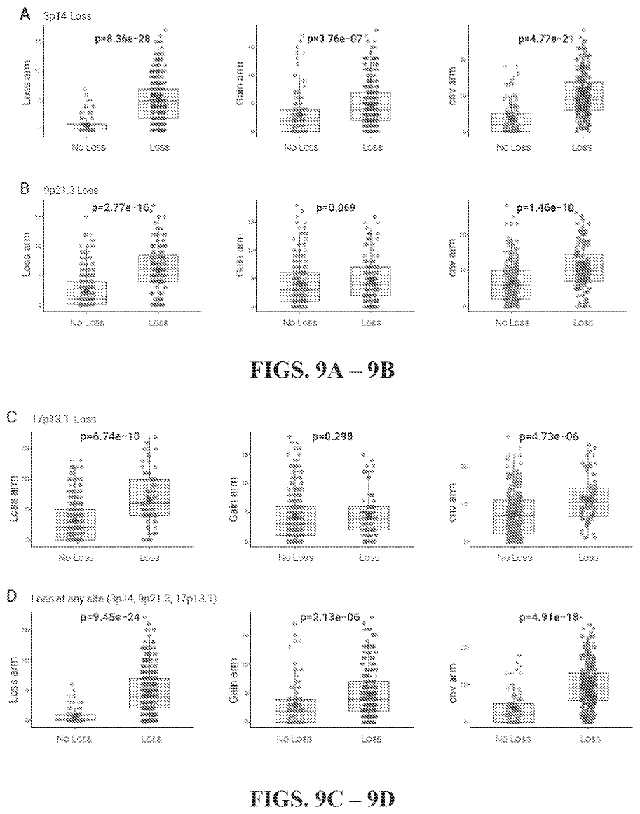

To define loss at 3p14, 9p21, or 17p13 loci, Applicant considered purity-adjusted CN measured in the same genomic location used to probe loss in precancers (using pathology- and ABSOLUTE-based purity estimates). Unless otherwise specified, loss of a genomic region (for example ‘9p21.3 loss’), refers to a loss at this region irrespective of the length of the deletion (e.g., whether focal or arm). In all cases, analyses were performed considering both specific-risk CN (e.g., 9p21.3 loss) as a binary variable (presence or absence of SCNA) and CN as a continuous variable (log-transformed CN level). 3p14, 9p21, and/or 17p13 loss (most associated with SCNA level and CN losses, FIG. 9), chr7 gain, and SCNA level (using cancer and precancer metrics) were associated with reduced CD3/CD8+ cell numbers, activation markers (GZMB, IFNG) and IS (FIG. 2 and FIG. 8).

Since individual SCNAs tended to co-occur (e.g., 3p14 loss and 9p21.3 loss, p=1.73E-26) and were associated with overall SCNA level, Applicant then determined the individual contribution of each major CN event in predicting immune marker and infiltrate parameter. Multivariable logistic regression was performed to predict the level of infiltrating CD3+ and CD8+ T cells, dividing the tumors into those containing high or low levels using predefined 35% and median cut offs. As predictive elements, CN metrics included those used in the precancer cohort—3p14, 9p21.3, and/or 17p13.1 loss, chr7 gain, and overall SCNA level. According to this model, 3p14 or 17p13.1 losses were not statistically significantly associated with CD3+ or CD8+ T-cell infiltration (FIG. 2B), IS, or GZMB/IFNG (data not shown). In contrast, 9p21.3 loss had a strong negative association with CD3+ and CD8+ T-cell infiltrates (β=−1.005, p=0.006, OR=0.366, 95% CI=0.178-0.744 for CD3; and β=−1.285, p=0.0004, OR=0.277, 95% CI=0.133-0.562 for CD8; FIG. 2B). Importantly, this robust association was independent of overall SCNA level, which was also statistically significantly negatively associated with CD3+ and CD8+ T-cell infiltrates (FIG. 2B). Although immune cold in univariate analysis (FIG. 8C), the chr7-tumor microenvironment signal was lost in multivariable analyses which included SCNA level (FIG. 2B). Neither 9p21.3 loss nor overall SCNA level was associated with CD68+ cells (FIG. 2C). Analyses using CN metric for major or minor loci loss and overall (precancer- and HNSC-defined) SCNA levels as a continuous variable (in addition to binary loss/gain classification based on a CN threshold) and with purity correction (using ABSOLUTE) gave similar results. 9p21.3 loss and SCNA level were identified as the two strongest predictors of T-cell depletion (data not shown). Without being bound by theory, size-effect analysis showed that 9p21.3 loss could explain 29.41% of the variance for CD3+ and 37.41% for CD8+ T cells (FIG. 2B). These results were similar when using 5-fold cross validation and adding other covariates, such as age, gender, TP53, and stage (table not shown).

Applicant notes that 3p14 loss was statistically significantly associated with immune-cold tumors in only one of the models tested here (continuous-variable model, using pathology-based estimate for purity correction). 3p14 loss associated with lower CD8+(β=0.583, p=0.007, OR=1.791, 95% CI=1.174-2.765) and CD3+ (β=0.585, p=0.003, OR=1.796, 95% CI=1.231-2.662) T cells and IS (β=0.601, p=0.005, OR=1.824, 95% CI=1.208-2.801) (data not shown). 3p14 loss association with immune markers (0-coefficient) was weaker than that of 9p21.3 loss in every analysis conducted here. The analysis of arm- versus focal-level events in the continuous-data model showed a 3p-arm loss association with low CD3+ (p=0.030; data not shown), and a trend for CD8+ (p=0.091, data not shown) cell infiltrates (using standardized CN values). Furthermore, 3p14 focal-only loss also showed a trend in the continuous-data model for CD3+ (p=0.078, data not shown) but not for CD8+ (p=0.15, data not shown) cells. Besides 3p14 loss, when using CD3+ and CD8+ T cells for the prediction, neither the association with 17p-arm nor 17p13.1-focal loss were statistically significant (data not shown).

To match the oral-precancer data by HNSC anatomic subsite, Applicant repeated the analyses of CD8+, CD3+, and CD68+ cells, IS, and arm/focal analysis conducted for all HPV-negative HNSC (data not shown), for the oral-cavity cancer-only subset in TCGA HPV-negative HNSC samples. The latter primary tumor samples were obtained from the alveolar ridge, buccal mucosa, floor of mouth, hard palate, lip, oral tongue or oral cavity (not otherwise specified). After this filter, the sample number decreased from 343 to 232, and the associations remained similar to that observed by considering all TCGA HPV-negative HNSC samples (data not shown; for oral-cavity only). Taken together, these data indicate that 9p loss in HPV-negative HNSC is linked to immune-exclusion, cold microenvironments. The 9p linkage, unlike 3p or 17p, was strong, specific, and consistent in every cancer-association analysis, whether it be univariate or multivariable, independent of threshold/cutoff and type of parameter (binary or continuous) or anatomic subsite. The linkage was confirmed in two purity-correction methods.

The association between 9p21.3 loss and immune-cold HPV-negative HNSC: driven mainly by entire 9p-arm loss. Loss at 9p21.3 can occur due to loss of the entire 9p arm, focal region at 9p21.3, or both (where arm and focal events are present in different chromosome copies). In all of the analyses described so far, SCNA was assessed at a specific genomic locus (e.g., 9p21.3) without information on deletion size. In precancer, estimates of CN level at 9p21.3 (by PCR) did not allow a distinction between arm or focal loss. However, in the context of HNSC data, this distinction was possible. Thus, the contribution of arm-versus focal-level loss to the association between 9p21.3 loss and immune infiltrates were determined. Specifically, 9p21.3 loss coming from arm-level events (9p arm loss) and 9p21.3 loss derived from focal-level only events was assessed. A profound effect of 9p arm-level loss was observed in predicting low CD3+ (β=−1.568, p=0.0008, OR=0.209, 95% CI=0.080-0.508; data not shown) and CD8+ (β=−1.795, p=0.0002, OR=0.166, 95% CI=0.061-0.413; data not shown) T-cell infiltrate, while the 9p21.3 focal-level event showed non-statistically significant decreases of CD8+ and CD3+ T-cell infiltration (FIG. 2D; data not shown).

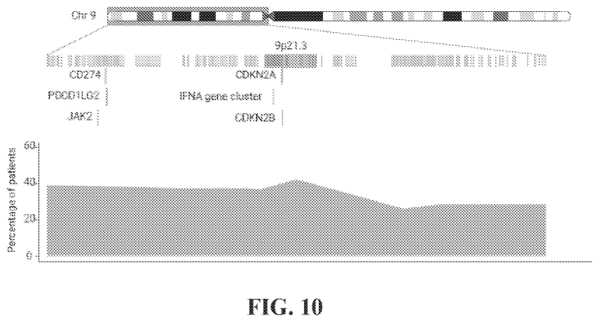

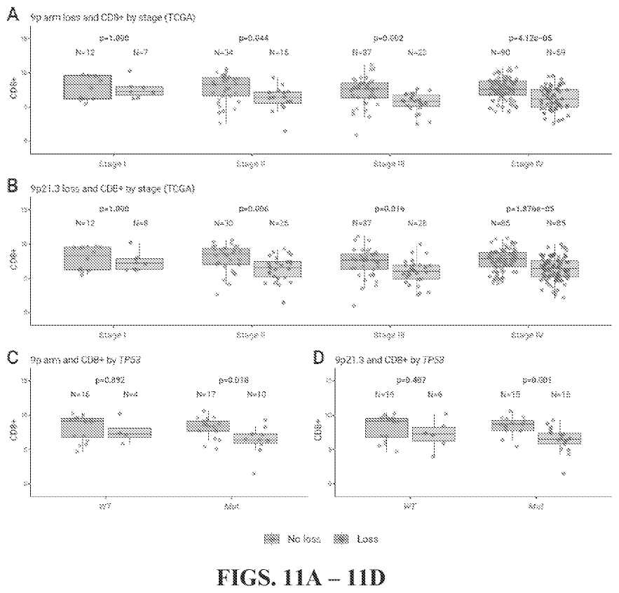

Similar results were obtained when considering deletion of specific candidate genes on 9p, specifically CDKN2A, IFNA, JAK2, and CD274, instead of the entire 9p21 or 9p24 regions. In all cases, arm-level deletion was a stronger predictor (in terms of β-coefficient and p-value) of CD3+ and CD8+ cells (data not shown), than focal deletion of these specific genes. Furthermore, in the Lasso classification method, 9p loss was the top scoring parameter selected to predict IS (p<0.001) across all possible arm-level losses or gains (data not shown). In this analysis, neither 3p nor 17p loss were found to be statistically significantly associated with CD3+ or CD8+ cell infiltrates. In analyses of whole-chromosome associations, chromosome-9 loss was the only event significantly associated with low CD3/CD8+ T-cell infiltrates and IS (p=0.008). This whole-chromosome association was seemingly due to the strong negative influence of 9p-arm loss on T-cell infiltrates, as the association of 9q loss with T-cell infiltrate was not statistically significant (data not shown). The phenotype is likely due to the cumulative effect of loss of many interacting or cooperating 9p genes outside of 9p21.3 (FIG. 10).

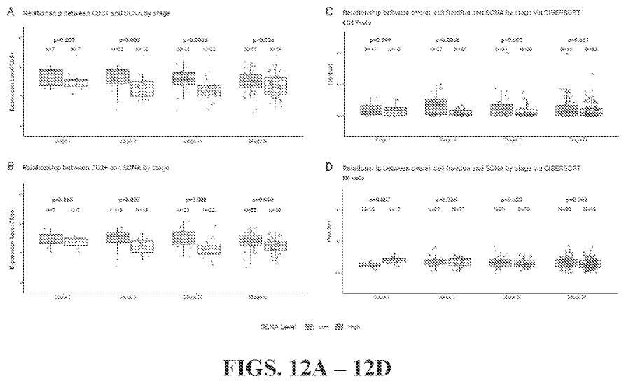

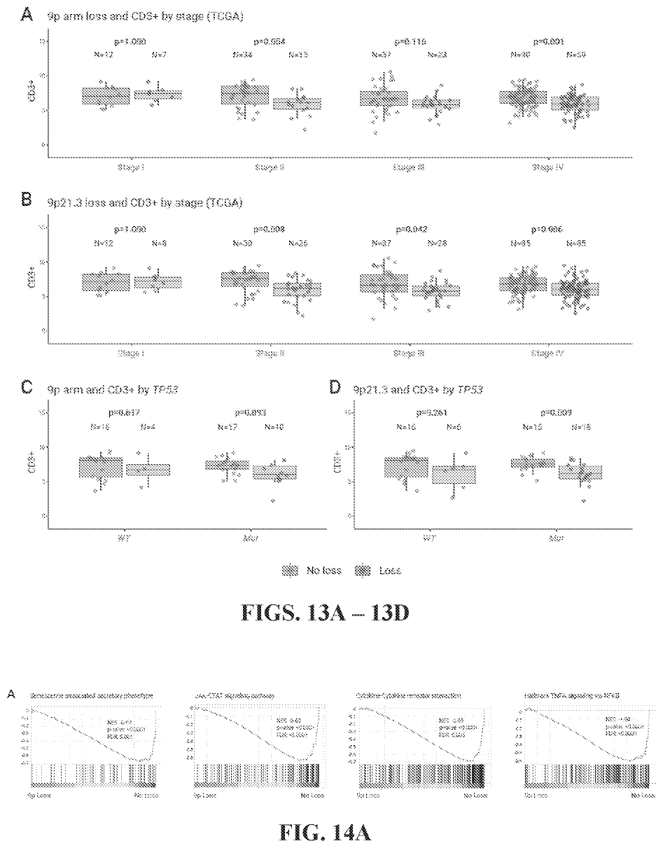

The association between 9p loss and immune infiltrate: influenced by stage and TP53 mutation. To test whether tumor microenvironment association was influenced at the early-invasive transition and by disease extent, Applicant stratified tumors by stage and found that the negative association of 9p21.3 loss, 9p arm-level loss, and SCNA level with CD3/CD8+ levels was no longer statistically significant in early HNSC (FIG. 11A, FIG. 11B, FIG. 12A, FIG. 12B and FIG. 13A, FIG. 13B; underlying data not shown). Since in dysplastic precancer, SCNA level was associated with a predominant CD3+ cell increase, Applicant inferred that specific immune-cell types may be associated with SCNA level in early-stage HNSC (data not shown). The CIBERSORT algorithm (30) was used to estimate the fraction of different cell types in the tumor microenvironment by CN metric. NK-cell levels were increased in SCNA-high tumors, specifically in early-stage primary disease (p=0.007; FIG. 12D). Chr7 gain did not show any stage-specific differences in NK or CD8+ cells. CIBERSORT analysis of CD8+ T-cell levels, however, revealed a stage-specific pattern opposite to that of NK cells, i.e., CD8+ levels were negatively associated with SCNA level (and 9p21 loss) in advanced (vs early) stage HNSC samples; a finding confirmed by logistic regression, purity-controlled analysis (FIG. 12C; underlying data not shown). The association of 9p21.3 or 9p loss with CD3+ and CD8+ T cells was stronger in TP53 mutant than wild-type tumors, especially in early-stage lesions (FIGS. 11C, 11D, 13C, and 13D; underlying data not shown).

Effect of SCNAs on immune infiltrate in HPV-positive HNSC. Applicant investigated SCNA frequencies and immune-cell associations in all 36 HPV-positive HNSCs available from TCGA. Compared to HPV-negative tumors, HPV-positive tumors showed a similar frequency of 17p13.1 loss but lower frequencies of both 3p14 and 9p21.3 loss (data not shown). Logistic regression for the prediction of CD3+, CD8+, and CD68+ cells showed that none of the CN parameters or metrics studied here were statistically significantly associated with any of these three cell types in HPV-positive tumors (data not shown).

### HPV-Negative HNSC Cell Lines

9p loss in HPV-negative HNSC cell lines was associated with decreased SASP, JAK-STAT, and cytokine pathways. Gene-expression profiles in HNSC cell lines (data not shown), both containing or not containing CN alterations associated with T-cell or IS suppression in TCGA HPV-negative HNSC analyses above were studied. Although in-vitro cell-line studies do not allow analyses of interactions with immune cells, they can identify cell-autonomous mechanisms that may mediate tumor-immune interactions. Applicant performed GSEA on transcriptomes of a set of 32 HPV-negative HNSC cell lines (data not shown), comparing cell lines with or without 9p-arm or 9p21.3 loss. Similar analyses were also performed considering 3p-arm or 3p14 loss. Among the top 10 pathways depleted in cell lines containing 9p-arm loss, seven were associated with immune processes or interactions. Three of the most significantly depleted pathways in cell lines containing 9p-arm loss were the Senescence-Associated Secretory Pathway (SASP) (FDR p<0.0001), Cytokine-Cytokine-Receptor Interaction (FDR=0.005), and JAK-STAT Signaling pathways (FDR=0.166; FIG. 3A; underlying data not shown). These pathways represent inter-related gene sets that encode for mostly secreted molecules that promote tumor-immune infiltration. A similar gene-set analysis showed that the top depleted Hallmark pathway was TNFA Signaling via NFkB (FDR<0.0001; FIG. 3A). In contrast, 3p14 or 3p loss were not associated with depletion of these or other pathways related to immune processes, but instead, associated with enrichment of Cytokine-Cytokine-Receptor Interaction and JAK-STAT Signaling (FDR=0.084 and 0.231, respectively) pathways, and pro-inflammatory molecule expression (data not shown).

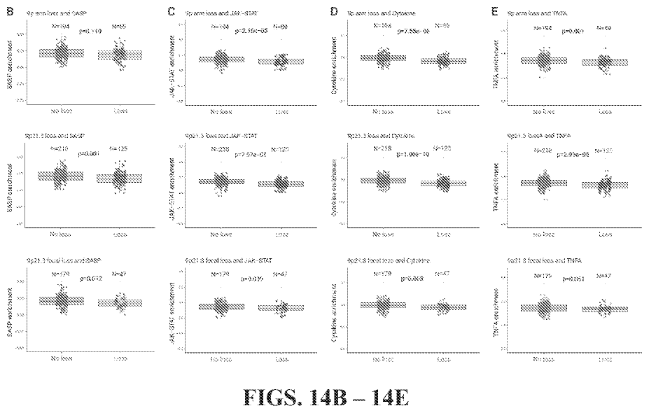

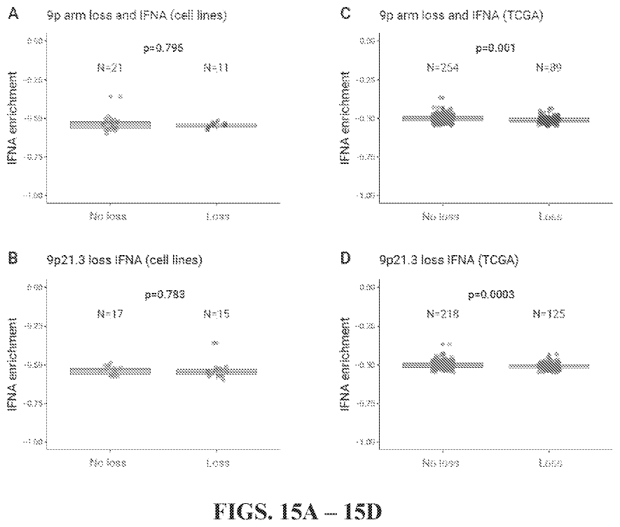

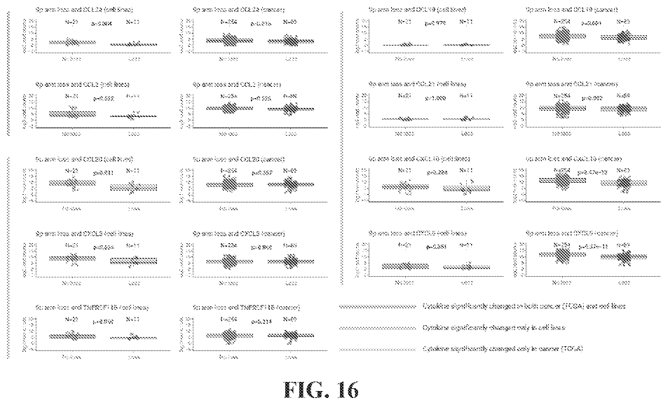

The expression of IFNα molecules was lower in tumors (TCGA) containing 9p21.3 loss, but not in cell lines harboring 9p21.3 or 9p loss, consistent with an extrinsic microenvironment effect (data not shown). JAK2 was also decreased in TCGA (but not cell lines) harboring 9p loss. Single-sample GSEA (ssGSEA) (31) analysis confirmed that 9p-arm loss was associated with suppressed SASP, JAK-STAT Signaling, Cytokine-Cytokine Receptor, and TNFA Signaling via NFkB pathways (FIGS. 3B-3E). Since the TCGA analysis above suggested that 9p21.3 loss contributes to immune-cold tumors promoted by 9p-arm deletion, Applicant examined this association in the cell lines. GSEA analysis comparing cell lines with or without loss at 9p21.3 showed profound depletion of SASP (FDR<0.0001), and a trend towards decreased JAK-STAT and Cytokine pathways (FIGS. 3B-E; underlying data not shown). These cell-line results suggest that 9p21.3 deletion contributes mainly to the cell-intrinsic SASP suppression, while deletion of the entire 9p arm is necessary for cell-line suppression of cytokines and other molecules related to Cytokine, JAK-STAT, and NFkB pathways. Compared to SASP, 9p-loss associated downregulation of the Cytokine-Cytokine-Receptor Interaction, JAK-STAT, and NFkB pathways were stronger in the tumor samples (FIGS. 14 and 15) than cell lines (FIG. 3). Immune-regulatory genes (belonging to one or more of the four pathways) downregulated in cell lines and tumors containing 9p loss were: CCL2, CCL24, CSF3R, KDR, IER5, IRF1, IL2RG, LTB, MCLI, SAT1, TNFSF10, and TNFRSF6B (FIG. 16). Molecules specifically decreased in tumors but not cell lines (FIG. 16), notably included CCL19 (log 2FC=−1.347, FDR=1.74E-06), CCL21 (log 2FC=−0.894, FDR=0.001), CXCL9 (log 2FC=−2.040, FDR=1.46E-15), and CXCL10 (log 2FC=−2.513, FDR=1.44E-20) (underlying data not shown).

Logistic regression model analysis was performed for the prediction of IS, taking into account SCNA level (as a covariate), to define potential candidate genes on 9p. Based on the logistic model, 84 genes were associated with IS (FDR<0.001) and among these, 42 showed significance (FDR<0.001) in at least two of the following parameters: correlation between RNA level and IS in tumors, between RNA level and DNA CN in tumors, or correlation between RNA and DNA CN in cell lines (data not shown). These genes, which contained JAK2, CDKN2A, and IFNα (32), included 22 genes in 9p13, 10 in 9p21, six on 9p22, and 10 genes on 9p24. New candidate genes identified in cell lines such as RANBP6, IL33, and the SUMO E3 ligase TOPORS, can decrease pro-inflammatory and pro-immunogenic molecules, and thus promote immune escape (FDR<0.0001, data not shown). In sum, these data indicate that in cancer-cell lines, 9p-arm deletion of key immunoregulatory genes can lead to T-cell depletion observed in tumors (e.g., CCL2, CCL24). Deletion of individual regions located on 9p, such as 9p21.3 or 9p24, were not sufficient to recapitulate the effects observed in cell lines harboring deletion of the entire arm, suggesting a cumulative effect of sets of genes located on this chromosome arm.

### Real-World Evidence Cohort

Chromosome-9p loss predicted survival of HPV-negative HNSC patients after immunotherapy. Based on the strong association between 9p loss and immune depletion in TCGA analyses above, Applicant examined whether there was a correlation between 9p loss and patient survival after immunotherapy in a real-world evidence (RWE) cohort. The independent de-identified RWE dataset contained genomic profiles annotated with clinical outcomes data (FIG. 16). Briefly, this cohort included 196 HPV-negative HNSC patients who received first- or second-line anti-PD-1 checkpoint therapy (pembrolizumab, nivolumab) or chemotherapy (with no prior or subsequent immunotherapy). Kaplan-Meier survival plots in this patient cohort with 9p loss treated with anti-PD-1 therapy, or chemotherapy alone, are shown in FIG. 4; 9p21, 9p13, and 9p24 chromosomal loss or gene deletion survival analyses are not shown. Within the context of the above PD-1 inhibitor therapy, median survival was longer in patients without 9p loss (HR=0.468, 95% CI=0.232-0.944, log-rank p=0.03) (FIG. 4A). To assess whether this survival difference reflected a generalized prognostic association (unrelated to PD-1 blockade), Applicant analyzed survival in patients treated with chemotherapy only (i.e., not treated with PD-1, checkpoint, inhibitors) and observed no difference between 9p loss vs no-loss groups (p=0.98) (FIG. 4B). These results indicated that 9p loss was a strong, specific predictive marker of clinical benefit from PD-1 inhibitors. In contrast, 3p, 17p, or 9p21.3 loss were not predictive (of anti-PD-1 therapy efficacy) (FIG. 4C), or prognostic in non-immune-checkpoint blockade (ICB) treated patients (data not shown).

PD-L1 and PD-L2 expression in tumor cells have been associated with benefits from nivolumab and pembrolizumab in HNSC patients (2-4, 26, 33). To assess whether the predictive effect of 9p-arm loss could be entirely attributed to loss of the PD-L1 (and/or PD-L2) gene (located in 9p24), survival in anti-PD-1-treated patients was evaluated according to the presence of PD-L1/-L2 gene deletion. Borderline predictive associations of PD-L1, -L2 and JAK2 deletion (at 9p24) were observed (data not shown), suggesting that additional gene alterations on 9p might contribute to resistance to PD-1 blockade. In regard to the strongest copy-number ICB-predictive marker in this study, patients with PD-L1-JAK2 co-deletion, had an inferior median survival vs those without this co-deletion (6 vs 19 months, p=0.007). 9p24 loss showed similar levels of anti-PD-1 therapy resistance (p=0.028) as observed with 9p-arm loss. As with 9p loss, deletions of PD-L1, PD-L2, JAK2, and 9p24, as well as PD-L1-JAK2 co-deletion were not prognostic in non-ICB treated patients (underlying data not shown). Tumor mutational burden (TMB; threshold of 10 mutations/megabase) was not associated with 9p loss or survival differences after PD-1 blockade (HR=0.762; 95% CI 0.423-1.372; p=0.364). None of the samples displayed microsatellite-instability-high (MSI-high).

### Discussion

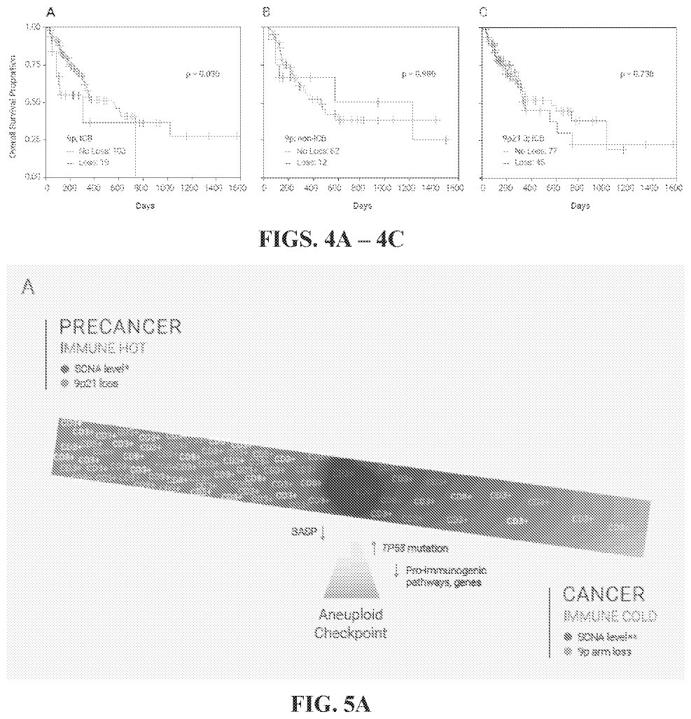

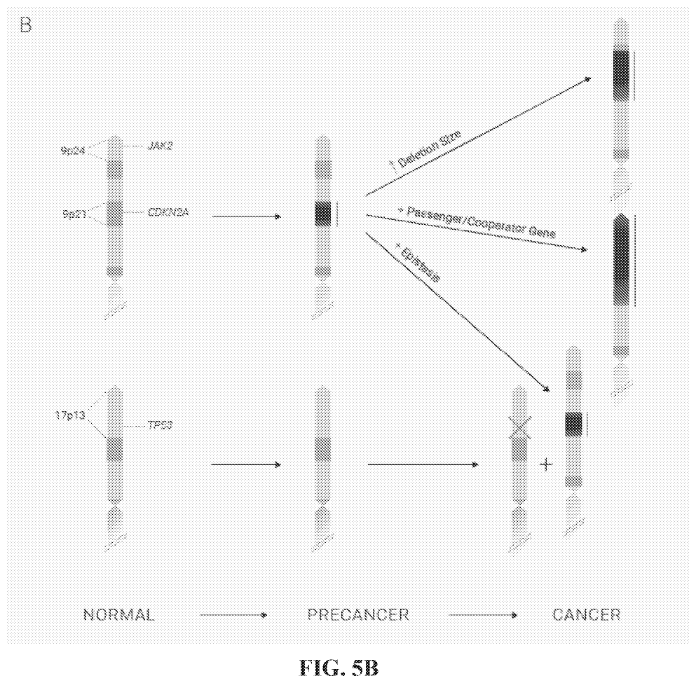

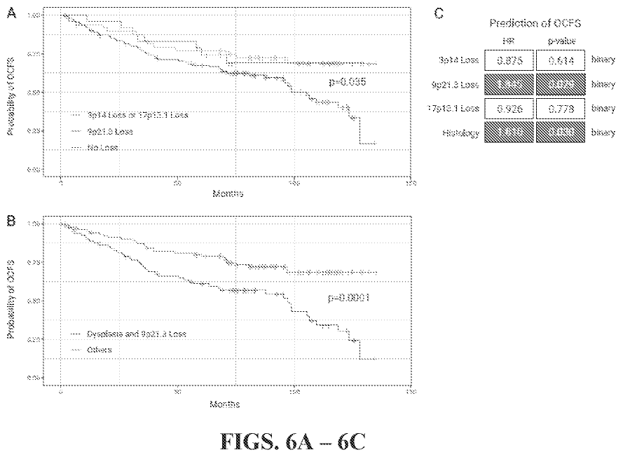

Aneuploid checkpoint and immune hot-to-cold switch in precancer-cancer transition. Applicant reports that chromosome-dosage imbalance contributes to an immune hot-to-cold switch during human HPV-negative head and neck tumorigenesis (FIG. 5A). Experimental-cell transfer of single chromosomes, or genome engineered focal alterations, trigger cytotoxic responses, regardless of the cell system or identity of the extra chromosome (24, 34, 35). Supporting these preclinical experiments, Applicant found that in clinical oral precancer lesions single-chromosome trisomy/tetrasomy correlated with increased CD3+ and CD8+ T-cell infiltrates, which contributed to overall SCNA level-hot associations. Without being bound by theory, CN-altered preneoplastic cells may elicit host recognition. The opposite SCNA-cold phenotype was observed in oral cancers, mostly with 9p-arm loss, marked by profound suppression of tumor-infiltrating cytotoxic T-cell number and activation markers (GZMB, IFNG, IS) (FIGS. 2B, 2D, and 8; underlying data not shown). This posits that during tumorigenesis, CN-generated immunogenic precancer cells acquire cold states, properties that allow their escape from immune-surveillance, -response, and eventual invasion and spread (24, 36, 37). Tumor evolution and selection of cells with further, complex, karyotypic imbalance and pivotal mutations (TP53) likely augment this process. These mechanisms have been observed under conditions of prolonged in vitro culture stress (24, 36) and in vivo immune pressure (37). These data uncover a striking context-dependent switch of SCNA influence on neoplastic microenvironments during oral precancer-to-cancer transition.

Role of 9p loss in immune evasion. This data indicates that 9p loss in oral precancer is the major genomic driver of cancer risk. Contributing to its pivotal function in tumorigenesis and malignant transformation, Applicant documented an integral role for 9p loss in immune escape (FIGS. 2B and 2D). In HPV-negative HNSC a profound influence was observed of 9p loss, but not 3p or 17p, on immune-exclusive, cold microenvironments. Without being bound by theory, these results also suggest that the previously reported association of 3p loss with decreased T-cell infiltrate and activation in HNSC specimens (38) may be due to marked co-occurrence of 3p14 loss and 9p21.3 loss (p=1.73E-26 in HPV-negative HNSC; data not shown). Furthermore, in cell lines not only was 3p loss not associated with immune depletion, but surprisingly in contrast, was associated with enrichment of immune-response and -pathway markers and metrics (data not shown), consistent with experimental 3p deletion in lung cells (24).

This data shows that a microenvironment switch is activated by arm-level 9p loss, with genes on 9p21.3 contributing, but not being sufficient, to drive immune evasion (FIGS. 2D, 3 and FIG. 14). Specific 9p loci relevant to immune response and escape include interferon-signaling pathway components, namely the IFNα-gene cluster (on 9p21.3) as well as key IFNγ-pathway gene JAK2 (on 9p24) (26) (FIG. 14, FIG. 15). In Applicant's experiments, the IFNα-gene cluster loss (on 9p21.3), postulated to promote tumor cell-intrinsic evasion in melanoma (32), does not appear to be operative in immune-cold HNSC with 9p loss. Without being bound by theory, Applicant's prediction-model implicates a cumulative effect of up to 40 potential candidate genes (data not shown), including CDKN2A (9p21.3), JAK2 (9p24), and several genes outside of 9p21.3 or 9p24, such as a prominent 22-gene cluster deletion in 9p13. Notable among the new candidates were RANBP6 and IL33, which can influence central processes involved in tumor microenvironment-cell recruitment and checkpoint-blockade efficacy by silencing STAT3 activity (39) and attracting immune cells to sites of tissue injury (40), respectively. A third possible culprit, TOPORS, can promote NFkB activity (41), potentially contributing to NFkB pathway suppression, T-cell depletion and escape after 9p loss.

From a mechanistic standpoint (FIG. 5), it is possible that chromosome loss needed for immune exclusion is larger in size and/or discontinuous. This concept, first suggested in a 3p-mapping study in lung squamous precancer, showed that focal chr3p loss was an early preneoplastic event, but its contributions to lung cancer resulted from progressive increases in size of 3p-arm loss (42). The study reported in 942) were extended to other chromosome-region losses in this disease (5q, 9p, 13q, 17p) (43), other human precancer types, and to model systems. The underlying basis for greater effects of larger chromosomal losses on neoplastic- and immune-transition, may be that targets within the region are augmented in effect by cooperating genes and regulatory elements elsewhere on the arm whose dosage titrates the effect of loss of primary target genes on the same chromosome (44, 45). It is also possible that 9p targets (the top arm-level event to predict low IS [data not shown]) are epistatic with genomic events on other chromosomes, thereby creating synthetic physiological effects, that exacerbate phenotype severity (46, 47) (FIG. 5B).

A well-established limitation of SCNA-microenvironment association studies involves tumor purity. Lower purity has been correlated with CN loss, microenvironment profile, and checkpoint blockade efficacy. Applicant therefore rigorously controlled for this important potential confounder in all genome-microenvironment association analyses conducted here. For each genomic site analyzed as a continuous and binary variable, pathology- and ABSOLUTE-based purity correction gave similar results, suggesting that Applicant's HNSC SCNA/genome-immune association findings were not confounded by purity issues. Furthermore, in this extensive statistical analyses, some reported associations were limited by marginal statistical significance within the context of multiple testing. Nevertheless, such results highlighted here were rigorously controlled for multiple testing (e.g., FDRs<0.001), consistent through various analyses, cut offs, and thresholds.

9p loss inhibits pro-immunogenic pathways. SCNA-pathway analyses provide insight into the mechanistic basis of 9p-loss driven immune evasion. Applicant found four interrelated pathways (SASP, Cytokine-Cytokine-Receptor Interaction, JAK-STAT Signaling, and TNFA Signaling via NFkB) statistically significantly decreased in primary tumors and cell lines harboring 9p loss. A key mutational difference between oral precancer and cancer is the substantially greater TP53 mutation frequency in the latter (9, 14, 48), which may remodel SCNA-generated immune-regulatory networks. TP53 plays a fundamental role in inducing SASP and senescence (49), and the TP53 target p21 (together with IL-1) is among the top genes upregulated after induction of chromosome missegregation and aneuploidy in TP53 wild-type cells (50). Chromosome instability and segregation errors, that can lead to aneuploidy, are known to activate cGAS-STING pathways (32) and in turn NFkB. Differential activation of the cGAS-STING pathway in CN altered cells may also account for SCNA-induced differential effects in precancer and cancer contexts. As with SASP, cell-intrinsic loss of cGAS or STING have been shown to have opposing roles in tumorigenesis. Early in precancer with focal 9p loss and limited SCNAs, cGAS-STING can lead to SCNA-detection and tumor-suppressive defense of transformation, then evolve immune-suppressive, tumor-promoting effects in later neoplastic stages characterized by progressive 9p-arm loss and karyotype complexity, which could inhibit or redirect downstream pathway promotion of immune evasion (21, 32, 37).

Additionally, the 9p21.3 region contains CDKN2A, which plays a central role in cell-cycle inhibition and SASP promotion in the presence of DNA damage and other cellular stresses, including aneuploidy (51, 52). 9p21.3 loss appeared important (and probably necessary) for depletion of SASP-related immune-regulatory molecules. In cell lines, 9p21.3 loss had a strong cell-intrinsic negative effect on SASP-pathway genes (data not shown;

FIGS. 3, 5, and FIG. 14). In fact, it was the one pathway decreased in cell lines with only focal 9p21.3 loss (data not shown). Decreased SASP pro-inflammatory molecule production, can impair T-cell recruitment (53). When TP53 is mutant, CDKN2A loss through 9p21.3 deletion may lead to decreased SASP, resulting in cold tumors. Consistent with these results, TP53-mutant, early-invasive disease with 9p21 deletion had a greater magnitude of CD3/CD8+ T-cell depletion compared to TP53 wild-type, 9p deleted counterparts. The association with 9p loss and mutant TP53 suppression of CD8 and CD3+ T cells was stronger with 9p21 than 9p arm loss (FIGS. 11C, 11D, 13C, and 13D; underlying data not shown).

In addition to SASPs, a profound decrease in chemokines CXCL9 and CXCL10 was found to be associated with 9p loss (54, 55). These IFNγ-inducible CXCR3 ligands, which attract type-1 cytokine producing effector T cells for cytolytic activity and further IFNγ production, are known to be secreted by tumor- and micro-environment-derived cells, such as dendritic cDC1 subset (56). IFNγ plays a prominent role in HNSC, and an IFNγ-signature was found to correlate with IS and anti-PD1 benefit in HNSC (29). Secretion of CXCR3 ligands greatly enhances tumor-antigen cross presentation essential for T-cell priming and activation (57). Additionally, Applicant found that 9p loss in TCGA (but not cell lines) was associated with a paucity of CCL19 and CCL21. Both may lead to a decrement in CD8+ effector T cells infiltrating the tumor site (58, 59). A lack of chemokine decrease in cell lines suggests the centrality of extracellular signals that limit chemokine production. These data suggest that 9p loss diminishes tumor-site effector T-cell recruitment, infiltration, and capacity for effective cell-mediated anti-tumor immunity. 9p loss orchestrates a chemokine-profile deficit, thus promoting a cold microenvironment, inhospitable to lymphocyte infiltration of the tumor. Since the pathways controlling immune-cell recruitment to the tumors are likely not cell-intrinsic, possibly involving secreted molecules, without being bound by theory, Applicant postulates that the presumably larger fraction of cells in precancer without arm deletions, may promote cell recruitment. This may partially mask the effect of 9p loss on cytokine and chemokine decrements.

Copy-number in early lesions and immune surveillance. Study of early-stage tumors, typically harboring one or a few arm/chromosome trisomies, may inform precancer karyotypic states, and shed light on the SCNA/immune-cell balance occurring during the pivotal invasive transition (36). In stage-specific analyses, the negative associations of 9p21.3, 9p arm-level loss, and SCNA level with CD3+ and CD8+ T-cell levels were weaker, and no longer statistically significant, in patients with early-stage HNSC in contrast to more advanced disease (FIGS. 11A, 11B, 12A-12C, and 13). Interestingly, SCNA level was associated with increased tumor-infiltrating NK-cell levels in stage-I HNSC samples (p=0.007, FIG. 12D). This is consistent with a previous pre-clinical study showing that aneuploidy in normal human cells can provoke NK-cell activation (21, 60). These stage-specific, cytotoxic-cell and TP53-mutation results could infer an aneuploid-immune switch emerging during an invasive transition inflection point, which strengthens with tumor evolution and progression. CN altered lesions, therefore must evolve properties and mechanisms to silence these immune responses and evade recognition by NK and other cytotoxic cells. Although CN burden in precursor lesions confers a high risk of malignant transformation, these SCNA-defined early lesions retained, to some extent, T- (FIGS. 1A-1D; underlying data not shown) and NK- (FIG. 12D) cell infiltration, possibly holding CN-altered pre-invasive lesions in check (61). Without being bound by theory, Applicant suggests that augmenting a residual microenvironment in this setting with anti-PD-1/PD-L1-centered prevention, however, may not have a high therapeutic yield, since PD-L1-independent evasion is expected to emerge as lesions with 9p loss obviate an aneuploid checkpoint. Applicant suggests, therefore, considering other immune therapies and combinations to intercept this high-risk precancer—notably T-reg-targeted/CTLA4 inhibitor-based strategies—and CD40 agonists. The latter, which can activate NK cells (62), was recently shown to prevent oral tumor development in mice (63).

The aneuploid-checkpoint concept proposed here can apply to cancers of sites other than the oral cavity, most notably lung squamous precancers/cancers. The latter track experimentally (24) and computationally with HPV-negative HNSC in pan-cancer genomic-SCNA association studies possibly reflecting shared co-evolution of immune evasion and neoplastic invasion (23, 24, 28, 64, 65). The latter was corroborated in a cross-sectional study of host detection in low-grade lung squamous precursors, through activation of resident immune cells, and escape through suppressive cells, networks, and signals, including interleukins and checkpoints, operative in high-grade disease (24, 66, 67). Furthermore, longitudinal studies of this precancer found that persistent, progressive lesions were associated with suppression of interferon-signaling, pathway gene expression and depletion of innate and adaptive cells (43). In these precancer studies, regressive high-grade lesions harbored more infiltrating immune cells than those that progressed to cancer (66). Consistent with lung squamous-cell carcinoma (LUSC) precursor studies, adaptive escape was already evident in lung adenocarcinoma precursors (5). Overall, lung carcinoma in situ (CIS) the direct, high-grade precursor to LUSC although preinvasive, had the full CN alteration profile displayed in invasive cancer, a finding mirrored in preinvasive studies of high-grade oral, breast, and lung adenocarcinoma in-situ (20, 68, 69). The most striking, consistent driver of SCNA-induced LUSC and HPV-negative HNSC was TP53 mutation (18). Virtually all lung CIS that progressed to invasive cancer were TP53 mutant and T-cell cold (66). The latter is consistent with Applicant's observation of greater influence of mutant TP53 on 9p-loss induced suppression of T-cell infiltration in early stage disease, and suggest potential of interception, targeting this pivotal event (70). In addition to lung cancer precursors (18), acquiring specific (e.g., large-scale 9p21.3, 17p, TP53) allelic losses and SCNA increases have been linked to immune-exclusive (cytotoxic-cell depleted) cold, and -suppressive (emergence of CD3/CD4+ T-regs; FIGS. 1A-1D; underlying data not shown) microenvironments in corresponding high-grade (and early invasive) progressive esophageal, colorectal, and breast lesions (19, 69, 71, 72).

Prediction of survival after cancer immunotherapy. Applicant identified 9p loss (FIG. 4A) as a highly specific predictive marker of anti-PD-1 clinical benefit in HPV-negative HNSC (p=0.03). Specific 9p alterations relevant to response and resistance to immunotherapy include deletion or impaired interferon-signaling pathway components, namely loss of IFNα genes (32) and IFNγ-pathway gene JAK2. The latter (JAK2 loss-of-function mutations) has been associated with anti-PD-1 and -CTLA4 resistance in melanoma (26). Although a consistent, marginally statistically significant pattern of JAK2-deletion associated anti-PD-1 resistance was found in this HNSC RWE, Applicant's data strongly support a dual-hit scenario where JAK2-PD-L1-co-deletion was associated with striking anti-PD-1 resistance with an HR of 0.436, 3-fold difference in median survival, p=0.007; a critical clinical impact that could be even further amplified by broader SCNA- (aneuploidy) induced cold tumors.

In stark contrast, 9p loss and PD-L1-JAK2 co-deletion in non-ICB chemotherapy-only treated patients, lack any prognostic impact, with HRs and p-values of near 1.0. Although CN gain was not associated with clinical benefit in ICB-treated melanoma patients, recurrent 9p loss was a statistically significant event in the no-clinical benefit subgroup (25). Consistent with Applicant's PD-L1-JAK2 co-deletion resistance findings, JAK2, PD-L1 (and PD-L2) (9p24.1) amplification, or CN gain, have been associated with opposite effects, namely anti-PD-1 benefit, primarily in metastatic melanoma (73, 74). Loss of 9p24 (and to a lesser degree 9p13) was associated with reduced survival trends after nivolumab or pembrolizumab (data not shown). Loss of 9p21 (IFNα gene set, CDKN2A deletion), 3p, and 17p were not predictive or prognostic in this cohort. Although TMB has been associated with anti-PD-1 benefit in limited HNSC studies (75, 76), Applicant was not able to detect such an effect in this 196-patient HNSC cohort (75, 76). Furthermore, TMB did not correlate with CN loss in Applicant's cohort, consistent with earlier HNSC (77) report, further indicating the discovery of a novel ICB-resistance mechanism. Direct therapeutic implications for patients with HPV-negative HNSC include to deprioritize single-agent PD-1 blockade and prioritize novel immune agents and combination strategies that could elicit immune response. SCNAs tend to differ by tissue type (22). However, these 9p loss and JAK2-PD-L1 co-deletion findings in anti-PD-1 treated HPV-negative HNSC can be applied to other tumors/sites and therapies, especially given the pivotal, broad role of JAK2 in cancer-cell sensitivity to IFNγ, impaired antigen presentation, T-cell sensitivity and evasion (26).

Applicant shows herein evidence from patient samples, in support of a CN-driven hot-to-cold switch in the precancer-invasion transition (FIG. 5). According to this model, CN-defined high-risk oral pre-invasive and early invasive lesions are immunogenic, suggesting possible clinical benefit of therapeutically augmenting the (presumably) still preserved immune surveillance in this setting. In parallel competing forces, CN-generated neoplastic evolution and immune escape requires acquisition of intrinsic properties to circumvent selective pressures from host surveillance, through further karyotypic, mutational, and other events, to overcome a pro-immunogenic aneuploid checkpoint and fuel tumor formation. Without being bound by theory, this microenvironment switch may be enabled through the combination of specific 9p-arm loss events with possible epistatic and other genomic events, such as CDKN2A-TP53 interactions leading to cell-intrinsic evasion through SASP and other mechanisms. A prominent role of 9p-arm deletion was identified (as compared to regional/gene hotspot deletions, or other SCNAs such as losses at 3p or 17p) in promoting depletion of cytotoxic cells (mainly CD8+ T cells), enriching suppressive cells (e.g., Tregs), molecules and networks operant in a subset of progressive lesions, and anti-PD-1 resistant tumors. The influence of 9p loss was independent of SCNA level, which was also significantly associated with decreased cytotoxic activity, likely driven by the cumulative, discontinuous loss of 9p-dosage clusters of established immune-regulatory genes at 9p21.3, 9p24, and possible new candidates on 9p13 and remote interactions (e.g., 17p13; FIG. 5B). These specific and broad CN-related phenomena and concepts are emerging in neoplasia, and other human diseases with aneuploid etiology, such as Down Syndrome, trisomy 21, phenotypes, e.g., activation of IFN signaling (36).

Genes Located on Chromosome 9p Arm

| Gene | Chromosome | |
|---|---|---|
| name | arm location | Cytoband |
| CNTNAP3 | 9p 13 | 9p13.1 |
| ZNF658 | 9p 13 | 9p13.1 |
| ZBTB5 | 9p 13 | 9p13.2 |
| GRHPR | 9p 13 | 9p13.2 |
| RNF38* | 9p 13 | 9p13.2 |
| FBXO10 | 9p 13 | 9p13.2 |
| DCAF10 | 9p 13 | 9p13.2 |
| ZCCHC7 | 9p 13 | 9p13.2 |
| EXOSC3 | 9p 13 | 9p13.2 |
| TOMM5 | 9p 13 | 9p13.2 |
| POLR1E | 9p 13 | 9p13.2 |
| MELK | 9p 13 | 9p13.2 |
| SHB | 9p 13 | 9p13.2 |
| NOL6 | 9p 13 | 9p13.3 |
| NFX1* | 9p 13 | 9p13.3 |
| BAG1 | 9p 13 | 9p13.3 |
| CHMP5 | 9p 13 | 9p13.3 |
| UBE2R2 | 9p 13 | 9p13.3 |
| UBAP2 | 9p 13 | 9p13.3 |
| UBAP1 | 9p 13 | 9p13.3 |
| DCAF12 | 9p 13 | 9p13.3 |
| UNC13B | 9p 13 | 9p13.3 |
| CCDC107 | 9p 13 | 9p13.3 |
| PIGO | 9p 13 | 9p13.3 |
| STOML2 | 9p 13 | 9p13.3 |
| VCP | 9p 13 | 9p13.3 |
| TESK1 | 9p 13 | 9p13.3 |
| FANCG | 9p 13 | 9p13.3 |
| DNAJB5 | 9p 13 | 9p13.3 |
| C9orf131 | 9p 13 | 9p13.3 |
| TLN1 | 9p 13 | 9p13.3 |
| GBA2* | 9p 13 | 9p13.3 |
| CREB3 | 9p 13 | 9p13.3 |
| CA9 | 9p 13 | 9p13.3 |
| RGP1 | 9p 13 | 9p13.3 |
| DCTN3 | 9p 13 | 9p13.3 |
| GALT* | 9p 13 | 9p13.3 |
| IL11RA | 9p 13 | 9p13.3 |
| SIGMAR1 | 9p 13 | 9p13.3 |
| NUDT2 | 9p 13 | 9p13.3 |
| HINT2 | 9p 13 | 9p13.3 |
| KIF24 | 9p 13 | 9p13.3 |
| SMU1 | 9p 21 | 9p21.1 |
| DNAJA1 | 9p 21 | 9p21.1 |
| B4GALT1 | 9p 21 | 9p21.1 |
| APTX | 9p 21 | 9p21.1 |
| TOPORS | 9p 21 | 9p21.1 |
| NDUFB6 | 9p 21 | 9p21.1 |
| DDX58 | 9p 21 | 9p21.1 |
| ACO1 | 9p 21 | 9p21.1 |
| MLLT3 | 9p 21 | 9p21.3 |
| ELAVL2 | 9p 21 | 9p21.3 |
| KLHL9 | 9p 21 | 9p21.3 |
| RPS6 | 9p 22 | 9p22.1 |
| DENND4C | 9p 22 | 9p22.1 |
| RRAGA | 9p 22 | 9p22.1 |
| HAUS6 | 9p 22 | 9p22.1 |
| CNTLN | 9p 22 | 9p22.2 |
| TTC39B | 9p 22 | 9p22.3 |
| PSIP1* | 9p 22 | 9p22.3 |
| SNAPC3 | 9p 22 | 9p22.3 |
| ZDHHC21 | 9p 22 | 9p22.3 |
| NFIB | 9p 23 | 9p23 |
| KIAA2026 | 9p 24 | 9p24.1 |
| ERMP1 | 9p 24 | 9p24.1 |
| AK3 | 9p 24 | 9p24.1 |
| KDM4C* | 9p 24 | 9p24.1 |
| RANBP6* | 9p 24 | 9p24.1 |
| IL33 | 9p 24 | 9p24.1 |
| UHRF2 | 9p 24 | 9p24.1 |
| CDC37L1 | 9p 24 | 9p24.1 |
| RCL1 | 9p 24 | 9p24.1 |
| RLN2 | 9p 24 | 9p24.1 |
| INSL6 | 9p 24 | 9p24.1 |
| RFX3 | 9p 24 | 9p24.2 |
| SLC1A1 | 9p 24 | 9p24.2 |
| VLDLR | 9p 24 | 9p24.2 |
| SMARCA2 | 9p 24 | 9p24.3 |
| CBWD1 | 9p 24 | 9p24.3 |
| KANK1 | 9p 24 | 9p24.3 |

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

1. D. Hanahan, R. A. Weinberg, Hallmarks of cancer: the next generation. Cell 144, 646-674 (2011).
2. B. Burtness et al., Pembrolizumab alone or with chemotherapy versus cetuximab with chemotherapy for recurrent or metastatic squamous cell carcinoma of the head and neck (KEYNOTE-048): a randomised, open-label, phase 3 study. Lancet 394, 1915-1928 (2019).
3. E. E. W. Cohen et al., Pembrolizumab versus methotrexate, docetaxel, or cetuximab for recurrent or metastatic head-and-neck squamous cell carcinoma (KEYNOTE-040): a randomised, open-label, phase 3 study. Lancet 393, 156-167 (2019).
4. R. L. Ferris et al., Nivolumab for Recurrent Squamous-Cell Carcinoma of the Head and Neck. The New England journal of medicine 375, 1856-1867 (2016).
5. K. Krysan et al., The immune contexture associates with the genomic landscape in lung adenomatous premalignancy. Cancer Res 10.1158/0008-5472.CAN-19-0153 (2019).
6. A. K. Chaturvedi et al., Oral Leukoplakia and Risk of Progression to Oral Cancer: A Population-Based Cohort Study. JNCI: Journal of the National Cancer Institute 10.1093/jnci/djz238 (2019).
7. E. E. Vokes, R. R. Weichselbaum, S. M. Lippman, W. K. Hong, Head and neck cancer. The New England journal of medicine 328, 184-194 (1993).
8. K. D. Shield et al., The global incidence of lip, oral cavity, and pharyngeal cancers by subsite in 2012. CA Cancer J Clin 67, 51-64 (2017).
9. J. Califano et al., Genetic Progression Model for Head and Neck Cancer: Implications for Field Cancerization. Cancer Research 56, 2488-2492 (1996).
10. L. Mao et al., Frequent microsatellite alterations at chromosomes 9p21 and 3p14 in oral premalignant lesions and their value in cancer risk assessment. Nat Med 2, 682-685 (1996).
11. W. N. Hittelman, Genetic instability in epithelial tissues at risk for cancer. Ann N Y Acad Sci 952, 1-12 (2001).
12. M. P. Rosin et al., Use of allelic loss to predict malignant risk for low-grade oral epithelial dysplasia. Clin Cancer Res 6, 357-362 (2000).
13. L. Zhang et al., Loss of heterozygosity (LOH) profiles—validated risk predictors for progression to oral cancer. Cancer Prev Res (Phila) 5, 1081-1089 (2012).
14. A. M. Gross et al., Multi-tiered genomic analysis of head and neck cancer ties TP53 mutation to 3p loss. Nat Genet 46, 939-943 (2014).
15. J. J. Lee et al., Predicting cancer development in oral leukoplakia: ten years of translational research. Clin Cancer Res 6, 1702-1710 (2000).
16. S. M. Lippman, W. K. Hong, Molecular markers of the risk of oral cancer. The New England journal of medicine 344, 1323-1326 (2001).
17. K. Krysan, L. M. Tran, S. M. Dubinett, Immunosurveillance and Regression in the Context of Squamous Pulmonary Premalignancy. Cancer Discov 10, 1442-1444 (2020).
18. V. H. Teixeira et al., Deciphering the genomic, epigenomic, and transcriptomic landscapes of pre-invasive lung cancer lesions. Nat Med 25, 517-525 (2019).
19. A. Spira et al., Leveraging premalignant biology for immune-based cancer prevention. Proceedings of the National Academy of Sciences of the United States of America 113, 10750-10758 (2016).
20. H. M. Wood et al., The genomic road to invasion—examining the similarities and differences in the genomes of associated oral pre-cancer and cancer samples. Genome Medicine 9, 53 (2017).
21. S. Santaguida et al., Chromosome Mis-segregation Generates Cell-Cycle-Arrested Cells with Complex Karyotypes that Are Eliminated by the Immune System. Dev Cell 41, 638-651 e635 (2017).
22. J. J. Bianchi, X. Zhao, J. C. Mays, T. Davoli, Not all cancers are created equal: Tissue specificity in cancer genes and pathways. Curr Opin Cell Biol 63, 135-143 (2020).
23. T. Davoli, H. Uno, E. C. Wooten, S. J. Elledge, Tumor aneuploidy correlates with markers of immune evasion and with reduced response to immunotherapy. Science 355 (2017).
24. A. M. Taylor et al., Genomic and Functional Approaches to Understanding Cancer Aneuploidy. Cancer Cell 33, 676-689 e673 (2018).
25. W. Roh et al., Integrated molecular analysis of tumor biopsies on sequential CTLA-4 and PD-1 blockade reveals markers of response and resistance. Science translational medicine 9 (2017).
26. T. E. Keenan, K. P. Burke, E. M. Van Allen, Genomic correlates of response to immune checkpoint blockade. Nat Med 25, 389-402 (2019).
27. M. S. Rooney, S. A. Shukla, C. J. Wu, G. Getz, N. Hacohen, Molecular and genetic properties of tumors associated with local immune cytolytic activity. Cell 160, 48-61 (2015).
28. D. Tamborero et al., A Pan-cancer Landscape of Interactions between Solid Tumors and Infiltrating Immune Cell Populations. Clin Cancer Res 24, 3717-3728 (2018).
29. M. Ayers et al., IFN-γ-related mRNA profile predicts clinical response to PD-1 blockade. J Clin Invest 127, 2930-2940 (2017).
30. A. M. Newman et al., Robust enumeration of cell subsets from tissue expression profiles. Nat Methods 12, 453-457 (2015).
31. A. Subramanian et al., Gene set enrichment analysis: a knowledge-based approach for interpreting genome-wide expression profiles. Proceedings of the National Academy of Sciences of the United States of America 102, 15545-15550 (2005).
32. S. F. Bakhoum, L. C. Cantley, The Multifaceted Role of Chromosomal Instability in Cancer and Its Microenvironment. Cell 174, 1347-1360 (2018).
33. J. H. Yearley et al., PD-L2 Expression in Human Tumors: Relevance to Anti-PD-1 Therapy in Cancer. Clin Cancer Res 23, 3158-3167 (2017).
34. L. Senovilla et al., An immunosurveillance mechanism controls cancer cell ploidy. Science 337, 1678-1684 (2012).
35. C. Viganó et al., Quantitative proteomic and phosphoproteomic comparison of human colon cancer DLD-1 cells differing in ploidy and chromosome stability. Mol Biol Cell 29, 1031-1047 (2018).
36. J. M. Sheltzer et al., Single-chromosome Gains Commonly Function as Tumor Suppressors. Cancer Cell 31, 240-255 (2017).
37. R. Tripathi, V. Modur, L. Senovilla, G. Kroemer, K. Komurov, Suppression of tumor antigen presentation during aneuploid tumor evolution contributes to immune evasion. Oncoimmunology 8, 1657374 (2019).
38. R. Mandal et al., The head and neck cancer immune landscape and its immunotherapeutic implications. JCI Insight 1, e89829 (2016).
39. B. Oldrini et al., EGFR feedback-inhibition by Ran-binding protein 6 is disrupted in cancer. Nat Commun 8, 2035 (2017).
40. B. C. L. Chan, C. W. K. Lam, L. S. Tam, C. K. Wong, IL33: Roles in Allergic Inflammation and Therapeutic Perspectives. Front Immunol 10, 364 (2019).
41. F. Renner, R. Moreno, M. L. Schmitz, SUMOylation-dependent localization of IKKepsilon in PML nuclear bodies is essential for protection against DNA-damage-triggered cell death. Mol Cell 37, 503-515 (2010).
42. Wistuba, II et al., High resolution chromosome 3p allelotyping of human lung cancer and preneoplastic/preinvasive bronchial epithelium reveals multiple, discontinuous sites of 3p allele loss and three regions of frequent breakpoints. Cancer Res 60, 1949-1960 (2000).
43. J. E. Beane et al., Molecular subtyping reveals immune alterations associated with progression of bronchial premalignant lesions. Nat Commun 10, 1856 (2019).
44. Y. Liu et al., Deletions linked to TP53 loss drive cancer through p53-independent mechanisms. Nature 531, 471-475 (2016).
45. W. Xue et al., A cluster of cooperating tumor-suppressor gene candidates in chromosomal deletions. Proceedings of the National Academy of Sciences 109, 8212-8217 (2012).
46. F. L. Muller et al., Passenger deletions generate therapeutic vulnerabilities in cancer. Nature 488, 337-342 (2012).
47. J. van de Haar et al., Identifying Epistasis in Cancer Genomes: A Delicate Affair. Cell 177, 1375-1383 (2019).
48. W. N. William et al., Genomic and transcriptomic landscape of oral pre-cancers (OPCs) and risk of oral cancer (OC). Journal of Clinical Oncology 37, 6009-6009 (2019).
49. A. Lujambio et al., Non-cell-autonomous tumor suppression by p53. Cell 153, 449-460 (2013).
50. S. Santaguida, E. Vasile, E. White, A. Amon, Aneuploidy-induced cellular stresses limit autophagic degradation. Genes Dev 29, 2010-2021 (2015).
51. G. A. Andriani et al., Whole Chromosome Instability induces senescence and promotes SASP. Sci Rep 6, 35218 (2016).
52. J. Y. Liu et al., Cells exhibiting strong p16 (INK4a) promoter activation in vivo display features of senescence. Proceedings of the National Academy of Sciences of the United States of America 116, 2603-2611 (2019).
53. W. Xue et al., Senescence and tumour clearance is triggered by p53 restoration in murine liver carcinomas. Nature 445, 656-660 (2007).
54. K. M. Perrott, C. D. Wiley, P. Y. Desprez, J. Campisi, Apigenin suppresses the senescence-associated secretory phenotype and paracrine effects on breast cancer cells. Geroscience 39, 161-173 (2017).
55. M. Yamane et al., Senescence-associated secretory phenotype promotes chronic ocular graft-vs-host disease in mice and humans. Faseb j 34, 10778-10800 (2020).
56. S. Spranger, D. Dai, B. Horton, T. F. Gajewski, Tumor-Residing Batf3 Dendritic Cells Are Required for Effector T Cell Trafficking and Adoptive T Cell Therapy. Cancer Cell 31, 711-723.e714 (2017).
57. S. K. Wculek et al., Dendritic cells in cancer immunology and immunotherapy. Nat Rev Immunol 20, 7-24 (2020).
58. S. Hillinger et al., EBV-Induced Molecule 1 Ligand Chemokine (ELC/CCL19) Promotes IFN-γ-Dependent Antitumor Responses in a Lung Cancer Model. The Journal of Immunology 171, 6457-6465 (2003).
59. C. E. Whyte et al., ACKR4 restrains antitumor immunity by regulating CCL21. Journal of Experimental Medicine 217 (2020).
60. R. W. Wang, S. Viganò, U. Ben-David, A. Amon, S. Santaguida, Aneuploid cells activate NF-κB to promote their immune clearance by NK cells. bioRxiv 10.1101/2020.06.25.172239, 2020.2006.2025.172239 (2020).
61. J. P. Foy et al., Immunological and classical subtypes of oral premalignant lesions. Oncoimmunology 7, e1496880 (2018).
62. R. Sartorius et al., Induction of human NK cell-mediated cytotoxicity by CD40 triggering on antigen presenting cells. Cell Immunol 221, 81-88 (2003).
63. J. A. Monteiro de Oliveira Novaes et al., Targeting of CD40 and PD-L1 Pathways Inhibits Progression of Oral Premalignant Lesions in a Carcinogen-induced Model of Oral Squamous Cell Carcinoma. Cancer Prev Res (Phila) 10.1158/1940-6207.Capr-20-0418 (2020).
64. J. D. Campbell et al., Genomic, Pathway Network, and Immunologic Features Distinguishing Squamous Carcinomas. Cell Rep 23, 194-212.e196 (2018).
65. R. S. Herbst, J. V. Heymach, S. M. Lippman, Lung Cancer. New England Journal of Medicine 359, 1367-1380 (2008).
66. A. Pennycuick et al., Immune Surveillance in Clinical Regression of Preinvasive Squamous Cell Lung Cancer. Cancer Discov 10, 1489-1499 (2020).
67. C. Mascaux et al., Immune evasion before tumour invasion in early lung squamous carcinogenesis. Nature 10.1038/s41586-019-1330-0 (2019).
68. J. Qian et al., Genomic Underpinnings of Tumor Behavior in In Situ and Early Lung Adenocarcinoma. American Journal of Respiratory and Critical Care Medicine 201, 697-706 (2020).
69. M. C. Abba et al., A Molecular Portrait of High-Grade Ductal Carcinoma In Situ. Cancer Res 75, 3980-3990 (2015).
70. C. M. Rudin et al., An attenuated adenovirus, ONYX-015, as mouthwash therapy for premalignant oral dysplasia. Journal of clinical oncology: official journal of the American Society of Clinical Oncology 21, 4546-4552 (2003).
71. A. Spira et al., Precancer Atlas to Drive Precision Prevention Trials. Cancer Res 77, 1510-1541 (2017).
72. A. Kouketsu et al., Regulatory T cells and M2-polarized tumour-associated macrophages are associated with the oncogenesis and progression of oral squamous cell carcinoma. Int J Oral Maxillofac Surg 48, 1279-1288 (2019).
73. J. Budczies, C. Denkert, B. Györffy, P. Schirmacher, A. Stenzinger, Chromosome 9p copy number gains involving PD-L1 are associated with a specific proliferation and immune-modulating gene expression program active across major cancer types. BMC Med Genomics 10, 74 (2017).
74. S. Gupta et al., JAK2, PD-L1, and PD-L2 (9p24.1) amplification in metastatic mucosal and cutaneous melanomas with durable response to immunotherapy. Hum Pathol 88, 87-91 (2019).
75. R. Cristescu et al., Pan-tumor genomic biomarkers for PD-1 checkpoint blockade-based immunotherapy. Science 362 (2018).
76. W. Li et al., Plasma-based tumor mutational burden (bTMB) as predictor for survival in phase III EAGLE study: Durvalumab (D)±tremelimumab (T) versus chemotherapy (CT) in recurrent/metastatic head and neck squamous cell carcinoma (R/M HNSCC) after platinum failure. Journal of Clinical Oncology 38, 6511-6511 (2020).
77. Y. P. Chen et al., Identification and validation of novel microenvironment-based immune molecular subgroups of head and neck squamous cell carcinoma: implications for immunotherapy. Ann Oncol 30, 68-75 (2019).
78. W. N. William, Jr. et al., Erlotinib and the Risk of Oral Cancer: The Erlotinib Prevention of Oral Cancer (EPOC) Randomized Clinical Trial. JAMA oncology 2, 209-216 (2016).
79. S. L. Carter et al., Absolute quantification of somatic DNA alterations in human cancer. Nat Biotechnol 30, 413-421 (2012).
80. C. H. Mermel et al., GISTIC2.0 facilitates sensitive and confident localization of the targets of focal somatic copy-number alteration in human cancers. Genome Biol 12, R41 (2011).
81. D. Aran, M. Sirota, A. J. Butte, Systematic pan-cancer analysis of tumour purity. Nat Commun 6, 8971 (2015).
82. M. Ghandi et al., Next-generation characterization of the Cancer Cell Line Encyclopedia. Nature 569, 503-508 (2019).

1. A method for treating cancer in a subject in need thereof, the method comprising administering PD-1/PD-L1 centered immunotherapy to the subject if a sample isolated from the patient comprising the cancer cell does not show 9p-arm loss.

2. A method for treating cancer in a subject in need thereof, the method comprising administering a therapy to the subject that does not comprise PD-1/PD-L1 centered immunotherapy to the subject if a sample isolated from the patient comprising the cancer cell shows 9p-arm loss.

3. A method for treating cancer in a subject in need thereof, the method comprising:
i) measuring 9p-arm loss in a cancer cell from the subject;
ii) determining a degree of 9p-arm loss; and
iii) administering PD-1/PD-L1 centered immunotherapy if the degree of 9p-arm loss does not show significant 9p-arm loss.

4. The method of claim 3, wherein step ii) comprises assessing 9p21.3 loss.

5. The method of claim 1, wherein the cancer is HPV-HNSC.

6. The method of claim 4, wherein the cancer is HPV-HNSC and 9p21.3 loss is associated with immune-cold tumor microenvironment (TME) signal.

7. The method of claim 1, wherein 9p21.3 loss comprises loss of entire 9p arm measured as loss of ≥70% of arm length.

8. The method of claim 7, further comprising assessing suppression of IFN-γ-inducible CXCL9/10 expression.

9. The method of claim 6, further comprising assessing CD8+ level in a sample isolated from the patient and wherein immune cold TME signal is indicated by a low CD8+ level.

10. The method of claim 4, further comprising assessing mutated TP53.

11. The method of claim 7, further comprising assessing cell-intrinsic senescence-associated secretory pathway (SASP) suppression.

12. The method of claim 4, wherein 9p21.3 loss comprises assessing deletion of one or more of the following genes: MLLT3; ELAVL2 or KLHL9.

13. The method of claim 3, wherein step ii) comprises assessing 9p22.1 loss.

14. The method of claim 13, wherein 9p22.1 loss comprises assessing deletion of one or more of the following genes: RPS6; DENND4C; RRAGA; or HAUS6.

15. The method of claim 3, wherein step ii) comprises assessing 9p22.2 loss.

16. The method of claim 15, wherein 9p22.2 loss comprises assessing deletion of the CNTLN gene.

17. The method of claim 3, wherein step ii) comprises assessing 9p22.3 loss.

18. The method of claim 17, wherein 9p22.3 loss comprises assessing deletion of one or more of the following genes: TTC39B; PSIP1; SNAPC3; or ZDHHC21.

19. The method of claim 3, wherein step ii) comprises assessing 9p23 loss or 9p24.1 loss.

20. The method of claim 19, wherein 9p23 loss comprises assessing deletion of NFIB gene.

21-46. (canceled)

47. A method for treating cancer in a subject in need thereof, the method comprising:
measuring gene deletion of JAK2 and PD-L1 in a cancer cell from the subject;
wherein when genetic studies of JAK2 and PD-L1 is not indicative of co-deletion of JAK2 and PD-L1, then the subject is administered PD-1/PD-L1 centered immunotherapy, and
wherein when genomic assessing of JAK2 and PD-L1 is indicative of co-deletion of JAK2 and PD-L1, then the subject is not administered PD-1/PD-L1 centered immunotherapy, and an alternative treatment is administered to the subject.

48. A method of inhibiting the growth or progression of cancer in a subject comprising
i) measuring 9p-arm and 9p21 loss in a precancer cell from the subject;
ii) determining a degree of 9p-arm loss; and
iii) administering to the subject treatment that does not comprise PD-1/PD-L1 centered immunotherapy if the degree of 9p-arm loss shows significant 9p-arm loss.

49. A method for predicting or determining whether a subject with cancer will respond to treatment with immunotherapy, the method comprising
i) measuring 9p-arm loss in a cancer cell from the subject;
ii) determining a degree of 9p-arm loss; and
iii) determining that the subject will respond to administering PD-1/PD-L1 centered immunotherapy if the degree of 9p-arm loss does not show significant 9p-arm loss.

50. A method for predicting or determining whether a type of cancer will respond to treatment with immunotherapy, the method comprising:
i) measuring 9p-arm loss in a cancer cell from the subject;
ii) determining a degree of 9p-arm loss; and
iii) determining that the type of cancer will respond to administering PD-1/PD-L1 centered immunotherapy if the degree of 9p-arm loss does not show significant 9p-arm loss.

51. (canceled)
